(19) United States
(12) Patent Application Publication
(10) Pub. No.: US 2023/0363826 A1
(43) Pub. Date: Nov. 16, 2023

(54) PRE-DOCKING POSE OPTIMIZATION

(71) Applicant: Auris Health, Inc., Redwood City, CA (US)

(72) Inventors: Geoffrey Robert Russell, San Jose, CA (US); Kieran Anthony Coghlan, Saratoga, CA (US)

(21) Appl. No.: 18/309,294

(22) Filed: Apr. 28, 2023

Related U.S. Application Data
(60) Provisional application No. 63/340,445, filed on May 10, 2022.

(51) Int. Cl. A61B 34/10; A61B 34/20; A61B 34/35; A61B 34/00; B25J 9/16

(52) U.S. Cl. CPC A61B 34/10; A61B 34/20; A61B 34/35; A61B 34/70; B25J 9/1666; B25J 9/1671; A61B 2034/105; A61B 2034/107; A61B 2034/2065

(57) ABSTRACT

Robotic medical systems may recommend poses for robotic arms based on patient and medical procedure information and communicate the recommended poses to users of the robotic medical systems. The robotic medical systems may include robotic arms and processor(s). The robotic medical systems may be configured to obtain images of a patient, simulate a medical procedure to identify collisions, and select poses based on a number of collisions. The robotic medical systems may provide information indicating the recommended poses.

### CROSS-REFERENCE TO RELATED APPLICATIONS

This application claims benefit of U.S. provisional Pat. application Serial No. 63/340,445 filed May 10, 2022 and entitled “PRE-DOCKING POSE OPTIMIZATION,” which is incorporated herein by reference in its entirety for all purposes.

### TECHNICAL FIELD

The systems and methods disclosed herein are directed to robotic medical systems, and more particularly to configuring robotically controlled arms of robotic medical systems for medical procedures.

### BACKGROUND

A robotically enabled medical system is capable of performing a variety of medical procedures, including both minimally invasive procedures, such as laparoscopy, and non-invasive procedures, such as endoscopy (e.g., bronchoscopy, ureteroscopy, gastroscopy, etc.).

Such robotic medical systems may include robotic arms configured to control the movement of medical tool(s) during a given medical procedure. In order to achieve a desired positioning of a medical tool, a robotic arm holding the medical tool can be placed into a particular pose during a set-up process or during teleoperation of the robotic medical system.

### SUMMARY

Surgical procedures can benefit from precise port locations and generally have a small tolerance for errors. For example, port placements may need to be within about 1.5 centimeters of an optimal position in large patients and within about 1 centimeter in smaller patients. Achieving such precise port placements satisfying small margins of error can be challenging.

In addition, different surgical procedures can involve different cannula and/or port locations. While conventional port planning often involves a doctor’s built-in knowledge of the types of procedures to be performed, this planning may not take into consideration the entire patient field and possible collisions that could occur between arms and external objects based on the port setup.

Robotic medical systems that include multiple robotic arms (e.g., four or more robotic arms) can provide a surgeon with more flexibility to perform medical procedures. They can also enable the development of novel surgical procedures and approaches. However, such complex systems require the robotic arms to be carefully positioned during setup so as to avoid intra-operative collisions and still be able to reach the target anatomy during surgery. In particular, there is a need for a robotic medical system that can identify precise locations for port(s) placement and corresponding optimum starting positions for the robotic arms.

As disclosed herein, a robotic medical system may be configured to select initial robotic arm poses and corresponding port location(s) for a given patient and procedures. In some embodiments, the selected initial robotic arm poses and corresponding port locations are recommended or optimized initial robotic arm poses and corresponding port locations. Some embodiments disclosed herein combine recommended port placement data with information about (i) patient presentation, such as height, weight, descriptions of the abdomen (thorax) size and shape, and surgical history (e.g., indicating internal organ changes or scarring); and (ii) the surgical procedure so as to generate a pre-docking pose of the robotic arm(s).

Some embodiments disclosed herein use a three-dimensional (3-D) scanner (e.g., a scanning unit) that is communicatively connected with the robotic medical system to determine the recommended initial robotic arm poses (e.g., pre-docking poses) and corresponding port location(s). The 3-D scanner can be included with (e.g., is a component of, forms a part of, etc.) the robotic medical system. In some configurations, the 3-D scanner can be a component of the robotic medical system that is located on a robotic arm, a patient support platform (e.g., bed), or a tower pendant of the robotic medical system, either as an integrated component or an attachment. In some other configurations, the 3-D scanner can be part of an instrument (e.g., a medical tool such as a laparoscope) that is held by a robotic arm of the robotic medical system. Alternatively, the 3-D scanner can be a separate accessory (e.g., a standalone scanner, a handheld scanner etc.) that is communicatively connected with the robotic medical system, and attached to (e.g., mounted on) a robotic arm, a patient support platform (e.g., bed), a tower pendant of the robotic medical system, or on a wall and/or ceiling of the operating room where the robotic medical system is located. The 3-D scanner can provide information such as patient view, patient characteristics, and/or locations of mounted accessories, which can be used by the robotic medical system (e.g., in combination with other information such as target procedure and/or anatomy) to determine recommended initial robotic arm poses (e.g., pre-docking poses) and corresponding port location(s) that are specifically tailored for the patient and/or medical procedure to be performed.

As disclosed herein, the 3-D scanner can be used to localize the patient relative to the robotic medical system. While a robotic medical system can determine the positions and/or orientations of the patient support platform and/or robotic arms of the robotic medical system (e.g., using sensors and/or encoders attached thereon), it can be a challenge for the robotic medical system to register other objects, such as the patient and mounted accessories that can include, and not limited to, a liver retractor, leg stirrups, arm boards, and anesthesia accessories, etc. Thus, localizing the patient relative to the robotic medical system with the 3-D scanner can provide to the robotic medical system information related to positions of the patient and/or mounted accessories.

As disclosed herein, the data from the 3-D scanner can be combined with other information / inputs, such as information about the target procedure and/or target anatomy, to generate the recommended initial robotic arm poses and corresponding port location(s). For example, the scan data (e.g., imaging data, 3-D imaging data, etc.) can include real-time patient information, such as patient size, patient characteristics (e.g., whether the patient is an amputee), and associate objects (e.g., if there is a feeding tube coming out of the patient this can demarcate a “keep out” zone). The robotic medical system can segment the patient into different sections (e.g., head, trunk, legs), and using the location of a marker (e.g., an abdomen of the patient) and/or relevant organ locations for a particular surgical procedure, the robotic medical system (e.g., via an algorithm) can recommend port placements and starting poses that would avoid or minimize collisions such as those between the patient and a robotic arm, between robotic arms, and/or between instruments, while maximizing available workspace during the procedure.

Accordingly, in one aspect, a robotic medical system includes a plurality of robotic arms; one or more processors; and memory storing instructions that, when executed by the one or more processors, cause the one or more processors to: (i) obtain imaging data of a patient; (ii) obtain information on a medical procedure for the patient; (iii) for each starting state of a plurality of starting states, simulate the medical procedure from the starting state to identify a number of collisions occurring with arms of the plurality of robotic arms, the simulating using the imaging data and the information on the medical procedure; (iv) select a first starting state of the plurality of starting states, the first starting state having a minimum number of collisions, where the first starting state identifies respective starting poses for each arm of the plurality of robotic arms; and (v) provide pose information for the first starting state to an operator of the robotic medical system.

In another aspect, a method for determining a starting state of a robotic system that includes a plurality of robotic arms includes: (i) obtaining imaging data of a patient; (ii) obtaining information on a medical procedure for patient; (iii) for each starting state of a plurality of starting states, simulating the medical procedure from the starting state to identify a number of collisions occurring with arms of the plurality of robotic arms, the simulating using the imaging data and the information on the medical procedure; (iv) selecting a first starting state of the plurality of starting states, the first starting state having a minimum number of collisions, where the first starting state identifies respective starting poses for each arm of the plurality of robotic arms; and (v) providing pose information for the first starting state to an operator of the robotic medical system.

The systems, methods, and devices of this disclosure each have several innovative aspects, no single one of which is solely responsible for the desirable attributes disclosed herein.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s), according to some embodiments.

FIG. 2 depicts further aspects of the robotic system of FIG. 1, according to some embodiments.

FIG. 3 illustrates an embodiment of the robotic system of FIG. 1 arranged for ureteroscopy, according to some embodiments.

FIG. 4 illustrates an embodiment of the robotic system of FIG. 1 arranged for a vascular procedure, according to some embodiments.

FIG. 5 illustrates an embodiment of a table-based robotic system arranged for a bronchoscopy procedure, according to some embodiments.

FIG. 6 illustrates an example system configured to stow robotic arm(s), according to some embodiments.

FIG. 7 illustrates an alternative embodiment of a table-based robotic system, according to some embodiments.

FIG. 8 illustrates an end view of the table-based robotic system of FIG. 7, according to some embodiments.

FIG. 9 illustrates an end view of a table-based robotic system with robotic arms attached thereto, according to some embodiments.

FIG. 10 illustrates an example instrument driver, according to some embodiments.

FIG. 11 illustrates an example medical instrument with a paired instrument driver, according to some embodiments.

FIG. 12 illustrates an alternative design for an instrument driver and instrument where the axes of the drive units are parallel to the axis of the elongated shaft of the instrument, according to some embodiments.

FIG. 13 illustrates an instrument having an instrument-based insertion architecture, according to some embodiments.

FIG. 14 illustrates an example controller, according to some embodiments.

FIG. 15 illustrates an example robotic system, according to some embodiments.

FIG. 16 illustrates another view of an example robotic system, according to some embodiments.

FIGS. 17A-17C illustrate different views of an example robotic arm, according to some embodiments.

FIGS. 18A-18C illustrate example cannula placement for different surgical procedures, according to some embodiments.

FIG. 19 illustrates an example workflow for generating pose and port information, according to some embodiments.

FIGS. 20A-20B illustrate example 3-D scanning and registration processes for a robotic medical system, according to some embodiments.

FIGS. 21A-21B illustrate an example process for identifying target anatomy locations, according to some embodiments.

FIG. 22A illustrates an example set of port locations for a medical procedure, according to some embodiments.

FIGS. 22B-22D illustrate example arm poses for the set of port locations of FIG. 22A, according to some embodiments.

FIG. 22E illustrates another example set of port locations for a medical procedure, according to some embodiments.

FIGS. 22F-22H illustrate example arm poses for the set of port locations of FIG. 22E, according to some embodiments.

FIGS. 23A and 23B are a flowchart illustrating an example method for selecting robotic arm poses, according to some embodiments.

FIG. 24 is a schematic diagram illustrating electronic components of a medical system, according to some embodiments.

### DETAILED DESCRIPTION

### 1 Overview

Aspects of the present disclosure may be integrated into a robotically enabled medical system capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

In addition to performing the breadth of procedures, the system may provide additional benefits, such as enhanced imaging and guidance to assist the physician. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations.

### A. Robotic System - Cart.

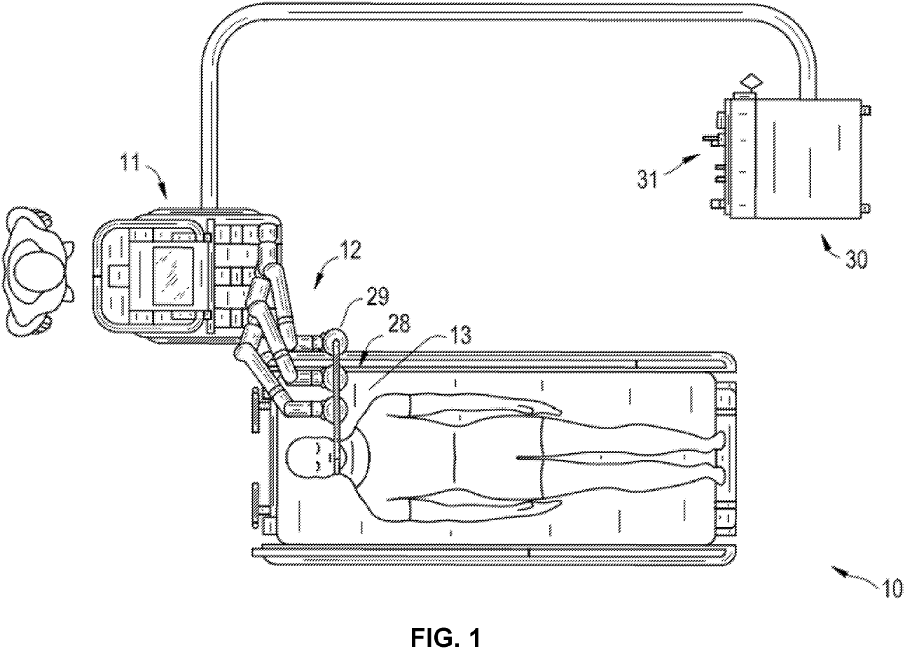

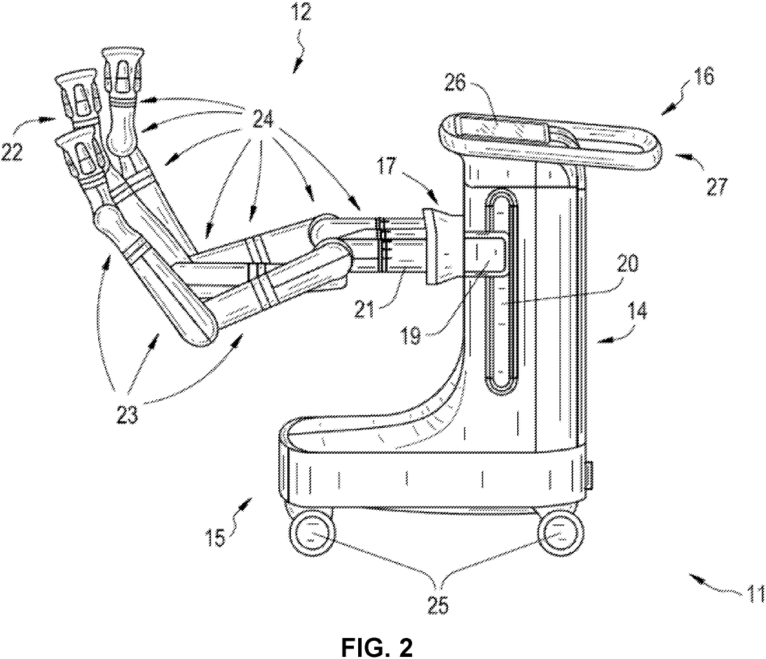

The robotically enabled medical system may be configured in a variety of ways depending on the particular procedure. FIG. 1 illustrates an embodiment of a cart-based robotically enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient’s upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope. FIG. 2 depicts an example embodiment of the cart in greater detail.

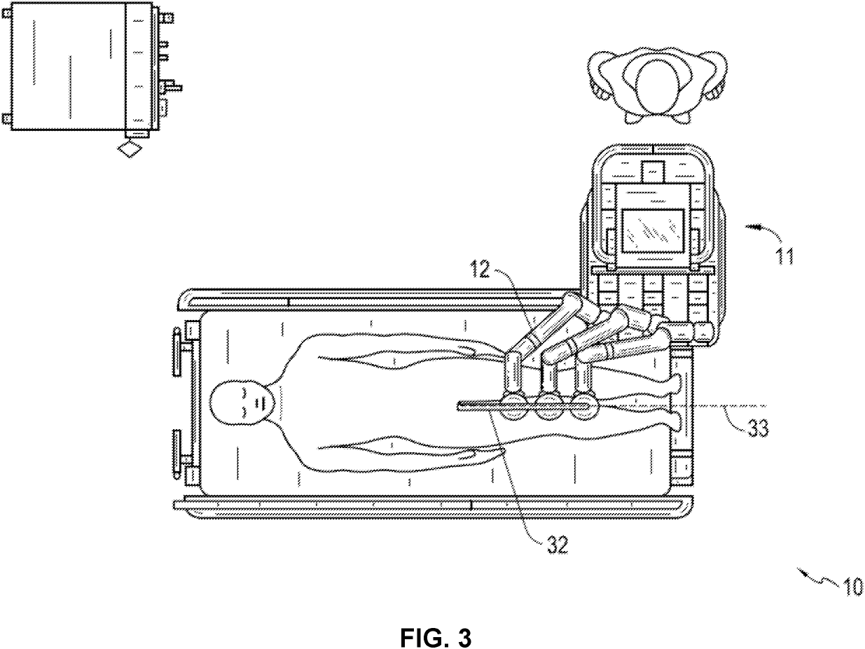

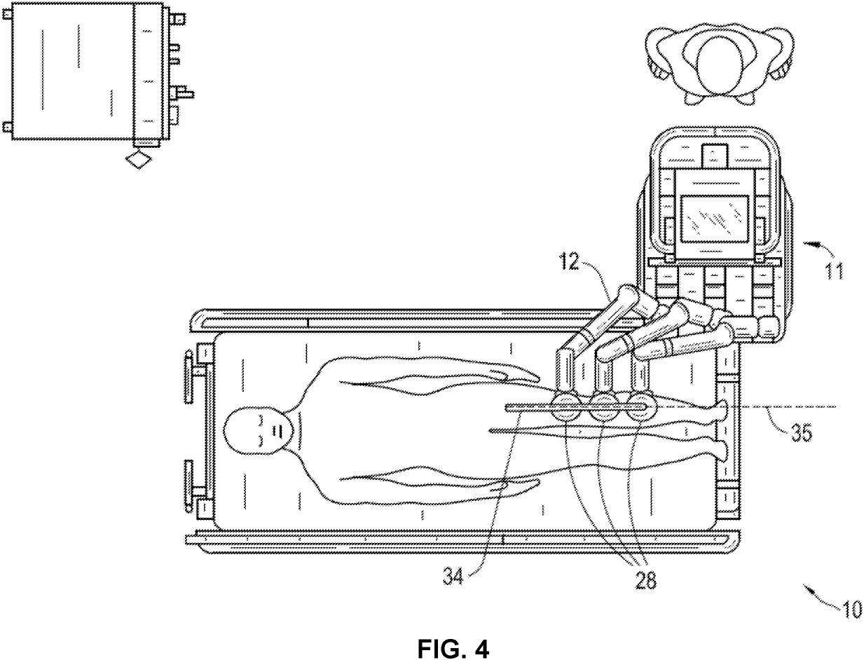

With continued reference to FIG. 1, once the cart 11 is properly positioned, the robotic arms 12 may insert the steerable endoscope 13 into the patient robotically, manually, or a combination thereof. As shown, the steerable endoscope 13 may include at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines (such as in FIGS. 3 and 4), and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient’s mouth.

The endoscope 13 may be directed down the patient’s trachea and lungs after insertion using precise commands from the robotic system until reaching the target destination or operative site. In order to enhance navigation through the patient’s lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to respect the potentially cancerous tissue. In some instances, diagnostic and therapeutic treatments can be delivered in separate procedures. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of the tower 30. In some embodiments, irrigation and aspiration capabilities may be delivered directly to the endoscope 13 through separate cable(s).

The tower 30 may also include support equipment for the sensors deployed throughout the robotic system 10. For example, the tower 30 may include optoelectronics equipment for detecting, receiving, and processing data received from the optical sensors or cameras throughout the robotic system 10. In combination with the control system, such optoelectronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in the system 10 are generally designed to provide both robotic controls as well as preoperative and real-time information of the procedure, such as navigational and localization information of the endoscope 13. When the console 31 is not the only console available to the physician, it may be used by a second operator, such as a nurse, to monitor the health or vitals of the patient and the operation of the system 10, as well as to provide procedure-specific data, such as navigational and localization information. In other embodiments, the console 31 is housed in a body that is separate from the tower 30.

FIG. 2 provides a detailed illustration of an embodiment of the cart 11 from the cart-based robotically enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage 17 at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of the robotic arms 12 to be angled in a variety of configurations.

The column 14 may internally include mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally include robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm 12. Each of the robotic arms 12 may have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and robotic arms 12 over the floor. Accordingly, the cart base 15 houses heavier components, such as electronics, motors, power supply, as well as components that either enable movement and/or immobilize the cart 11. For example, the cart base 15 includes rollable wheel-shaped casters 25 that allow for the cart 11 to easily move around the room prior to a procedure. After reaching the appropriate position, the casters 25 may be immobilized using wheel locks to hold the cart 11 in place during the procedure.

Positioned at the vertical end of the column 14, the console 16 allows for both a user interface for receiving user input and a display screen (or a dual-purpose device such as, for example, a touchscreen 26) to provide the physician user with both preoperative and intra-operative data. Potential preoperative data on the touchscreen 26 may include preoperative plans, navigation and mapping data derived from preoperative computerized tomography (CT) scans, and/or notes from preoperative patient interviews. Intra-operative data on display may include optical information provided from the tool, sensor and coordinate information from sensors, as well as vital patient statistics, such as respiration, heart rate, and/or pulse.

FIG. 3 illustrates an embodiment of a robotically enabled system 10 arranged for ureteroscopy. In a ureteroscopic procedure, the cart 11 may be positioned to deliver a ureteroscope 32, a procedure-specific endoscope designed to traverse a patient’s urethra and ureter, to the lower abdominal area of the patient. As shown, the cart 11 may be aligned at the foot of the table to allow the robotic arms 12 to position the ureteroscope 32 for direct linear access to the patient’s urethra. From the foot of the table, the robotic arms 12 may insert the ureteroscope 32 along the virtual rail 33 directly into the patient’s lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

FIG. 4 illustrates an embodiment of a robotically enabled system 10 similarly arranged for a vascular procedure. In a vascular procedure, the system 10 may be configured such that the cart 11 may deliver a medical instrument 34, such as a steerable catheter, to an access point in the femoral artery in the patient’s leg. As in a ureteroscopic procedure, the cart 11 may be positioned towards the patient’s legs and lower abdomen to allow the robotic arms 12 to provide a virtual rail 35 with direct linear access to the femoral artery access point in the patient’s thigh / hip region. After insertion into the artery, the medical instrument 34 may be directed and inserted by translating the instrument drivers 28. Alternatively, the cart may be positioned around the patient’s upper abdomen in order to reach alternative vascular access points, such as, for example, the carotid and brachial arteries near the shoulder and wrist.

### B. Robotic System - Table.

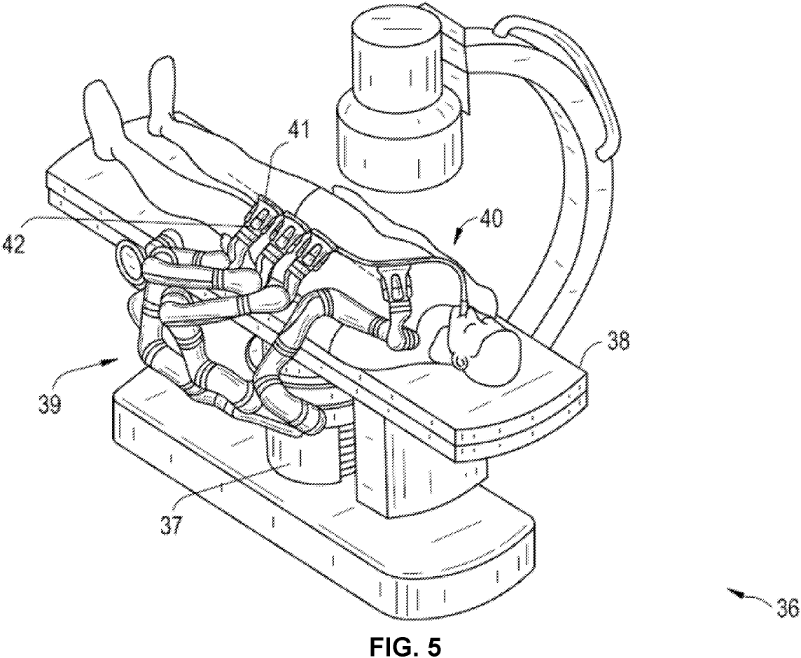

Embodiments of the robotically enabled medical system may also incorporate the patient’s table. Incorporation of the table may reduce the amount of capital equipment within the operating room by removing the cart, which allows greater access to the patient. FIG. 5 illustrates an embodiment of such a robotically enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient’s upper abdominal area by placing the emitter and detector around the table 38.

As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient.

The system 36 may also include a tower (not shown) that divides the functionality of the system 36 between the table and the tower to reduce the form factor and bulk of the table. As in earlier disclosed embodiments, the tower may provide a variety of support functionalities to the table, such as processing, computing, and control capabilities, power, fluidics, and/or optical and sensor processing. The tower may also be movable to be positioned away from the patient to improve physician access and de-clutter the operating room. The tower may also include a master controller or console that provides both a user interface for user input, such as keyboard and/or pendant, as well as a display screen (or touchscreen) for preoperative and intra-operative information, such as real-time imaging, navigation, and tracking information. In some embodiments, the tower may also contain holders for gas tanks to be used for insufflation.

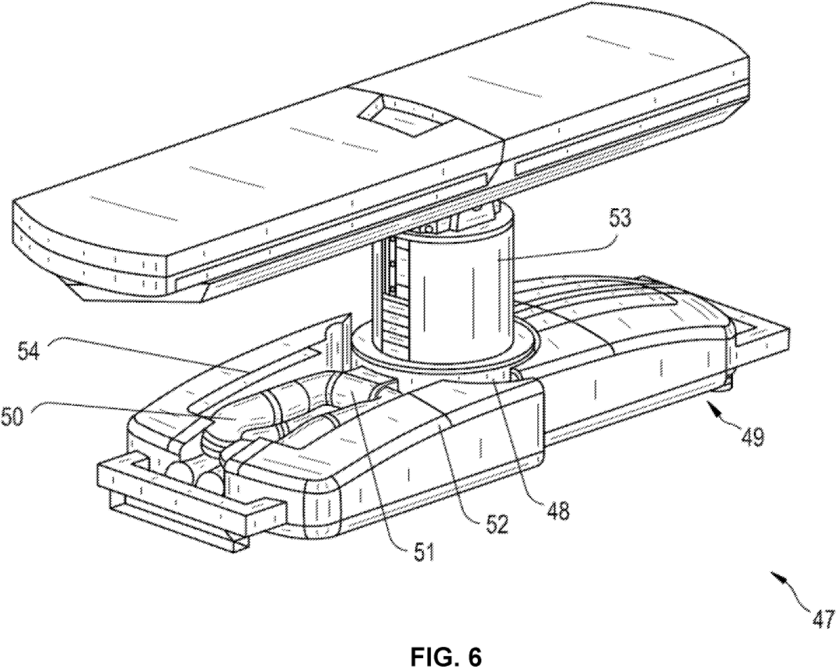

In some embodiments, a table base stows and stores the robotic arms when not in use. FIG. 6 illustrates a system 47 that stows robotic arms in an embodiment of the table-based system. In the system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use.

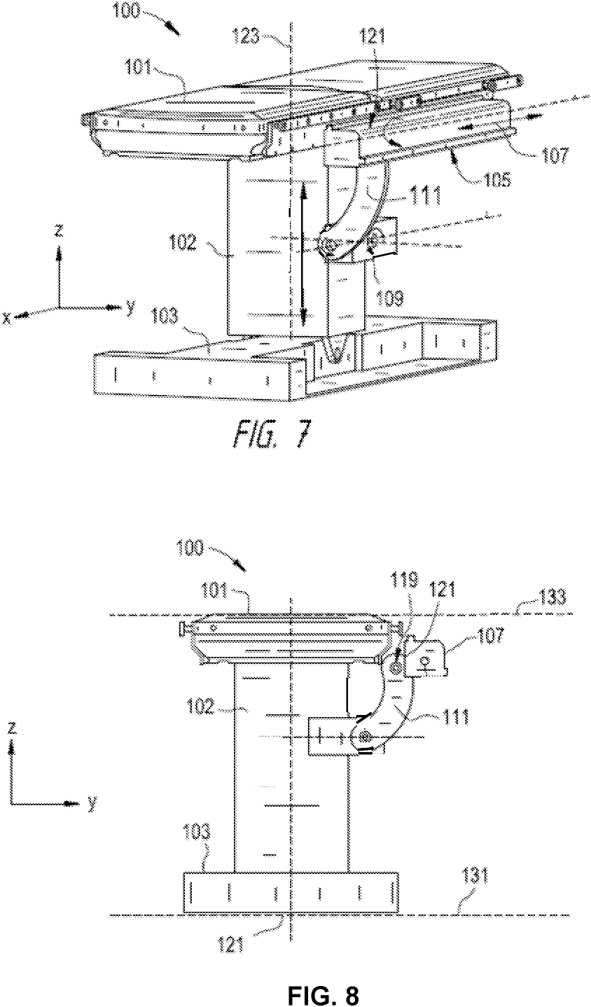

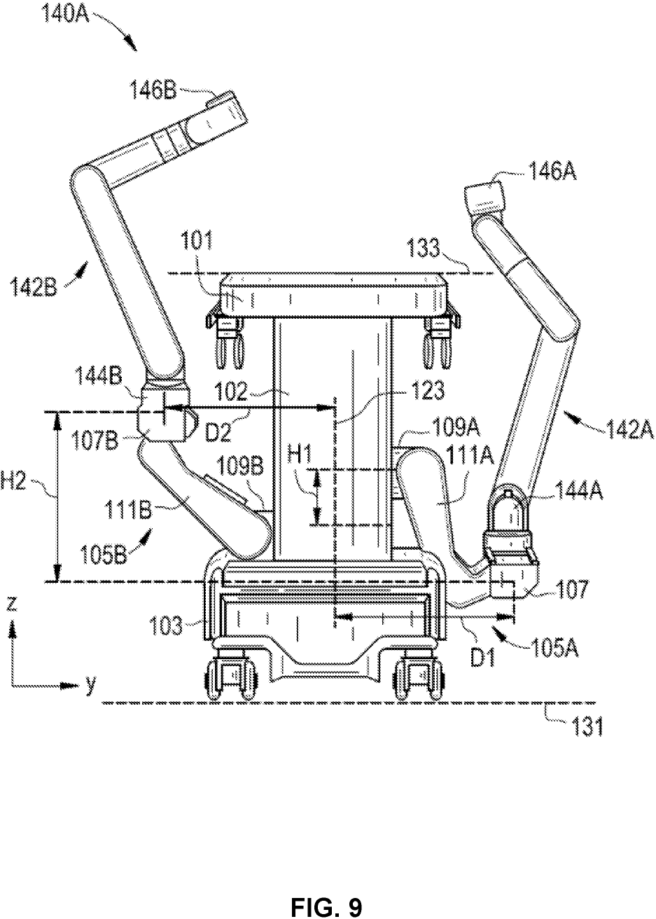

FIGS. 7 and 8 illustrate isometric and end views of an embodiment of a table-based surgical robotics system 100. The surgical robotics system 100 includes one or more adjustable arm supports 105 that can be configured to support one or more robotic arms (see, for example, FIG. 9) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 7 and 8, the arm support 105 is configured with four degrees of freedom, which are illustrated with arrows in FIG. 7. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 7 and 8 can include a table supported by a column 102 that is mounted to a base 103. The base 103 and the column 102 support the table 101 relative to a support surface. A floor axis 131 and a support axis 133 are shown in FIG. 8.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

FIG. 9 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B includes an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system’s robotic arms include (i) an instrument driver (alternatively referred to as “instrument drive mechanism” or “instrument device manipulator”) that incorporates electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician’s staff. In contrast, the instrument drivers need not be changed or sterilized, and may be draped for protection.

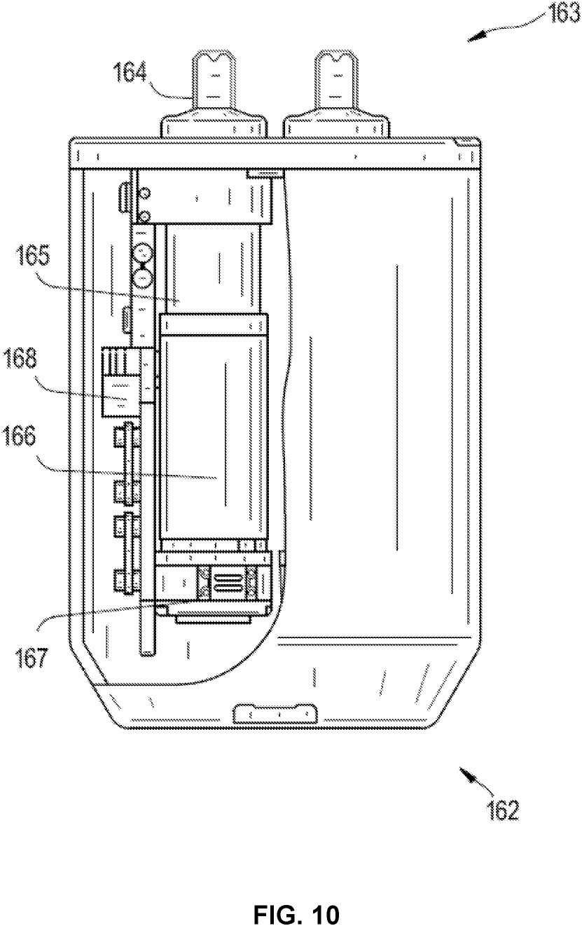

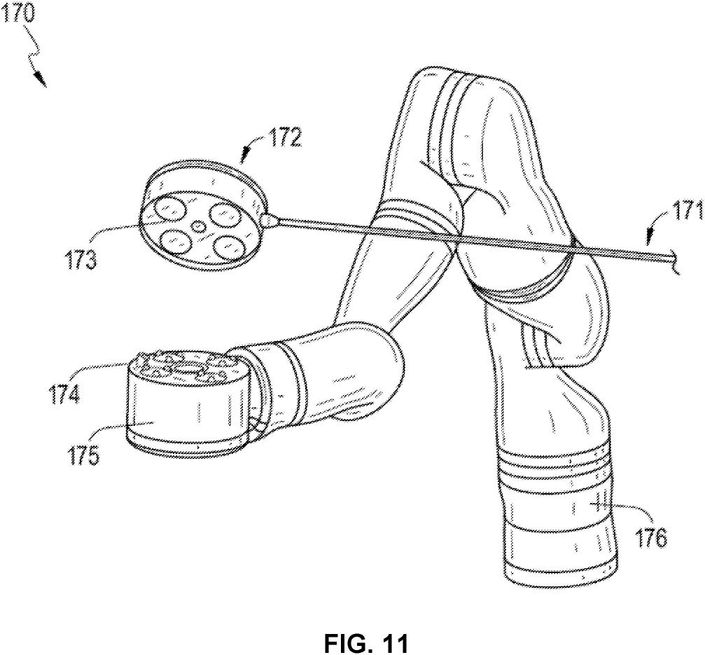

FIG. 10 illustrates an example instrument driver. Positioned at the distal end of a robotic arm, instrument driver 162 includes of one or more drive units 163 arranged with parallel axes to provide controlled torque to a medical instrument via drive shafts 164. Each drive unit 163 includes an individual drive shaft 164 for interacting with the instrument, a gear head 165 for converting the motor shaft rotation to a desired torque, a motor 166 for generating the drive torque, an encoder 167 to measure the speed of the motor shaft and provide feedback to the control circuitry, and control circuitry 168 for receiving control signals and actuating the drive unit. Each drive unit 163 being independent controlled and motorized, the instrument driver 162 may provide multiple (four as shown in FIG. 11) independent drive outputs to the medical instrument. In operation, the control circuitry 168 would receive a control signal, transmit a motor signal to the motor 166, compare the resulting motor speed as measured by the encoder 167 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may include a series of rotational inputs and outputs intended to be mated with the drive shafts of the instrument driver and drive inputs on the instrument. Connected to the sterile adapter, the sterile drape composed of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

FIG. 11 illustrates an example medical instrument with a paired instrument driver. Like other instruments designed for use with a robotic system, medical instrument 170 includes an elongated shaft 171 (or elongate body) and an instrument base 172. The instrument base 172, also referred to as an “instrument handle” due to its intended design for manual interaction by the physician, may generally include rotatable drive inputs 173, e.g., receptacles, pulleys, or spools, that are designed to be mated with drive outputs 174 that extend through a drive interface on instrument driver 175 at the distal end of robotic arm 176. When physically connected, latched, and/or coupled, the mated drive inputs 173 of instrument base 172 may share axes of rotation with the drive outputs 174 in the instrument driver 175 to allow the transfer of torque from drive outputs 174 to drive inputs 173. In some embodiments, the drive outputs 174 may include splines that are designed to mate with receptacles on the drive inputs 173.

The elongated shaft 171 is designed to be delivered through either an anatomical opening or lumen, e.g., as in endoscopy, or a minimally invasive incision, e.g., as in laparoscopy. The elongated shaft 171 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 174 of the instrument driver 175. When designed for endoscopy, the distal end of a flexible elongated shaft may include a steerable or controllable bending section that may be articulated and bent based on torque received from the drive outputs 174 of the instrument driver 175.

Torque from the instrument driver 175 is transmitted down the elongated shaft 171 using tendons along the shaft 171. These individual tendons, such as pull wires, may be individually anchored to individual drive inputs 173 within the instrument handle 172. From the handle 172, the tendons are directed down one or more pull lumens along the elongated shaft 171 and anchored at the distal portion of the elongated shaft 171, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 173 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 171, where tension from the tendon cause the grasper to close.

In endoscopy, the tendons may be coupled to a bending or articulating section positioned along the elongated shaft 171 (e.g., at the distal end) via adhesive, control ring, or other mechanical fixation. When fixedly attached to the distal end of a bending section, torque exerted on drive inputs 173 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 171 to allow for controlled articulation in the desired bending or articulable sections.

In endoscopy, the elongated shaft 171 houses a number of components to assist with the robotic procedure. The shaft may include of a working channel for deploying surgical tools (or medical instruments), irrigation, and/or aspiration to the operative region at the distal end of the shaft 171. The shaft 171 may also accommodate wires and/or optical fibers to transfer signals to/from an optical assembly at the distal tip, which may include of an optical camera. The shaft 171 may also accommodate optical fibers to carry light from proximally located light sources, such as light emitting diodes, to the distal end of the shaft.

At the distal end of the instrument 170, the distal tip may also include the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera. In the example of FIG. 11, the drive shaft axes, and thus the drive input axes, are orthogonal to the axis of the elongated shaft.

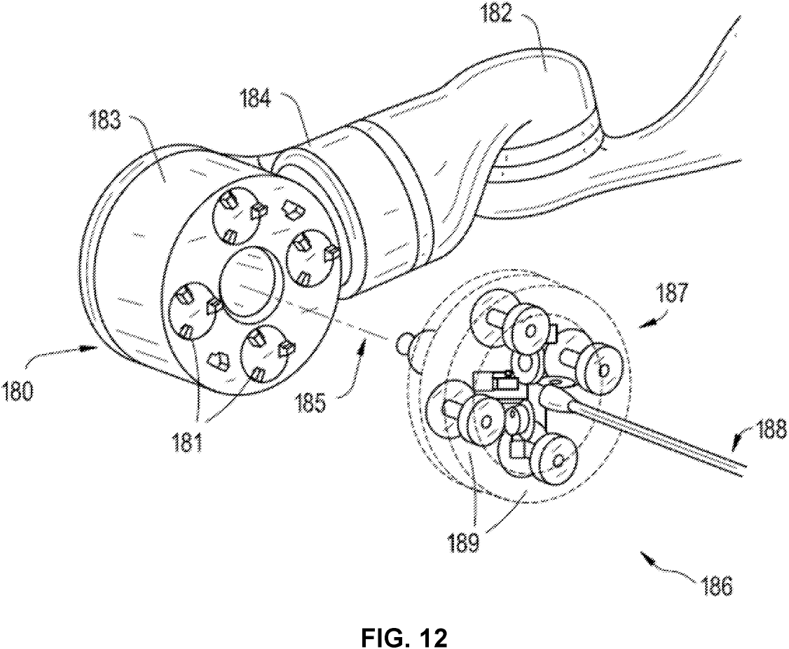

FIG. 12 illustrates an alternative design for an instrument driver and instrument where the axes of the drive units are parallel to the axis of the elongated shaft of the instrument. As shown, a circular instrument driver 180 includes four drive units with their drive outputs 181 aligned in parallel at the end of a robotic arm 182. The drive units, and their respective drive outputs 181, are housed in a rotational assembly 183 of the instrument driver 180 that is driven by one of the drive units within the assembly 183. In response to torque provided by the rotational drive unit, the rotational assembly 183 rotates along a circular bearing that connects the rotational assembly 183 to the non-rotational portion 184 of the instrument driver. In other embodiments, the rotational assembly 183 is responsive to a separate drive unit that is integrated into the non-rotatable portion 184, and thus not in parallel to the other drive units. The rotational mechanism 183 allows the instrument driver 180 to rotate the drive units, and their respective drive outputs 181, as a single unit around an instrument driver axis 185.

Like earlier disclosed embodiments, an instrument 186 may include an elongated shaft portion 188 and an instrument base 187 (shown with a transparent external skin for discussion purposes) comprising a plurality of drive inputs 189 (such as receptacles, pulleys, and spools) that are configured to receive the drive outputs 181 in the instrument driver 180. Unlike prior disclosed embodiments, instrument shaft 188 extends from the center of instrument base 187 with an axis substantially parallel to the axes of the drive inputs 189, rather than orthogonal as in the design of FIG. 11.

When coupled to the rotational assembly 183 of the instrument driver 180, the medical instrument 186, comprising instrument base 187 and instrument shaft 188, rotates in combination with the rotational assembly 183 about the instrument driver axis 185. Since the instrument shaft 188 is positioned at the center of instrument base 187, the instrument shaft 188 is coaxial with instrument driver axis 185 when attached. Thus, rotation of the rotational assembly 183 causes the instrument shaft 188 to rotate about its own longitudinal axis.

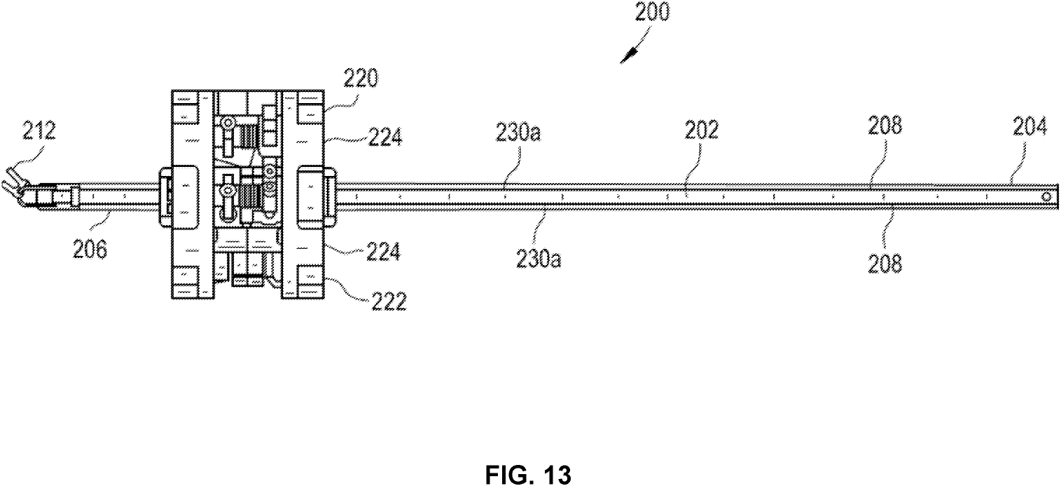

FIG. 13 illustrates an instrument having an instrument-based insertion architecture in accordance with some embodiments. The instrument 200 can be coupled to any of the instrument drivers discussed above. The instrument 200 includes an elongated shaft 202, an end effector 212 connected to the shaft 202, and a handle 220 coupled to the shaft 202. The elongated shaft 202 includes a tubular member having a proximal portion 204 and a distal portion 206. Manipulation of one or more cables 230a (e.g., via an instrument driver) results in actuation of the end effector 212.

The instrument handle 220, which may also be referred to as an instrument base, may generally include an attachment interface 222 having one or more mechanical inputs 224, e.g., receptacles, pulleys, or spools, that are designed to be reciprocally mated with one or more torque couplers on an attachment surface of an instrument driver.

In some embodiments, the instrument 200 includes a series of pulleys or cables that enable the elongated shaft 202 to translate relative to the handle 220. In other words, the instrument 200 itself includes an instrument-based insertion architecture that accommodates insertion of the instrument, thereby minimizing the reliance on a robot arm to provide insertion of the instrument 200. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

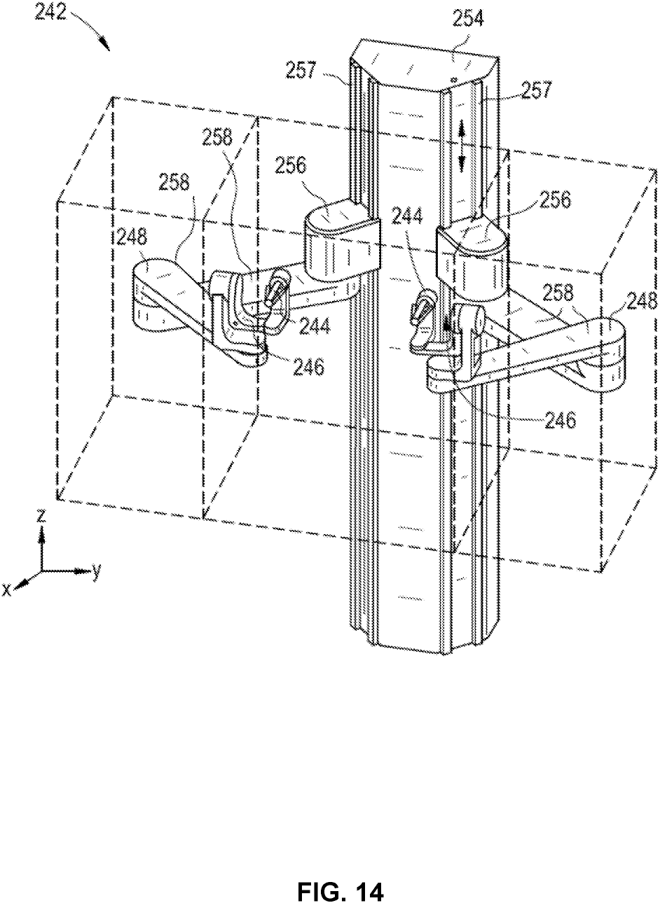

FIG. 14 is a perspective view of an embodiment of a controller 242. In the illustrated embodiment, the controller 242 is configured to allow manipulation of two medical instruments and includes two handles 244. Each of the handles 244 is connected to a gimbal 246. Each gimbal 246 is connected to a positioning platform 248.

As shown in FIG. 14, each positioning platform 248 includes a SCARA arm (selective compliance assembly robot arm) 258 coupled to a column 254 by a prismatic joint 256. The prismatic joints 256 are configured to translate along the column 254 (e.g., along rails 257) to allow each of the handles 244 to be translated in the z-direction, providing a first degree of freedom. The SCARA arm 258 is configured to allow motion of the handle 244 in an x-y plane, providing two additional degrees of freedom.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

As discussed above, the robotic systems discussed herein may be designed to incorporate a combination of one or more of the technologies above. The robotic system’s computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2 Pose Optimization for Robotic Systems

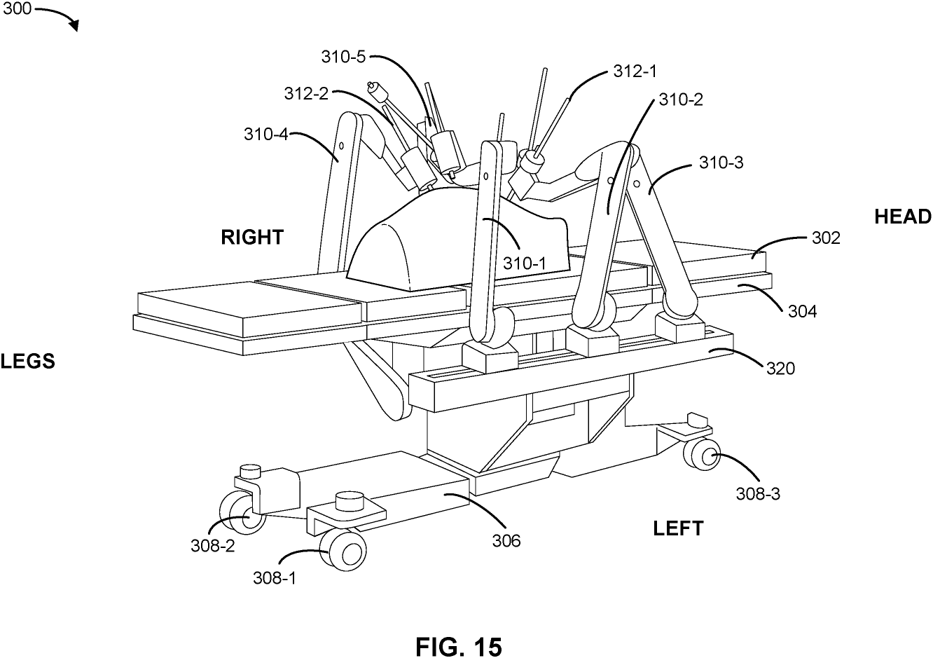

FIG. 15 illustrates a robotic medical system 300 according to some embodiments. In some embodiments, the robotic medical system 300 is a robotic surgery system. In the example of FIG. 15, the robotic medical system 300 includes a patient support platform 302 (e.g., a patient platform, a table, a bed, etc.). The two ends along the length of the patient support platform 302 are respectively referred to as “head” and “leg”. The two sides of the patient support platform 302 are respectively referred to as “left” and “right.” The patient support platform 302 includes a support 304 (e.g., a rigid frame) for the patient support platform 302.

The robotic medical system 300 also includes a base 306 for supporting the robotic medical system 300. The base 306 includes wheels 308 (casters) that allow the robotic medical system 300 to be easily movable or repositionable in a physical environment. In some embodiments, the wheels 308 are omitted from the robotic medical system 300 or are retractable, and the base 306 rests directly on the ground or floor. In some embodiments, the wheels 308 are replaced with feet.

The robotic medical system 300 includes one or more robotic arms 310. The robotic arms 310 can be configured to perform robotic medical procedures as described above with reference to FIGS. 1-14. Although FIG. 15 shows five robotic arms 310 (e.g., arms 310-1 through 310-5), it should be appreciated that the robotic medical system 300 may include any number of robotic arms, including less than five or six or more.

In accordance with some embodiments, the robotic medical system 300 also includes one or more arm supports 320 (e.g., a bar or an adjustable bar) that support the robotic arms 310. Each of the robotic arms 310 is supported on, and movably coupled to, an arm support 320, by a respective base joint of the robotic arm. In some embodiments, as described previously, the arm support 320 can provide several degrees of freedom, including lift, lateral translation, tilt, etc. In some embodiments, each of the robotic arms 310 and/or the adjustable arm supports 320 is also referred to as a respective kinematic chain.

FIG. 15 shows three robotic arms 310-1, 310-2, and 310-3 supported by the arm support 320 that is in the field of view of the figure. The two remaining robotic arms 310-4 and 310-5 are supported by another bar that is located across the other length of the patient support platform 302.

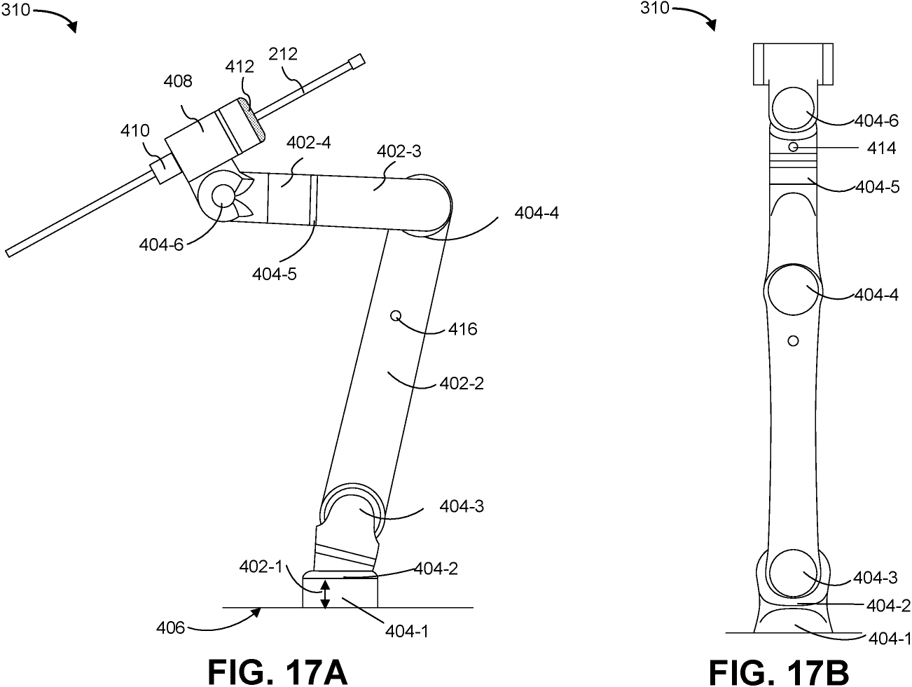

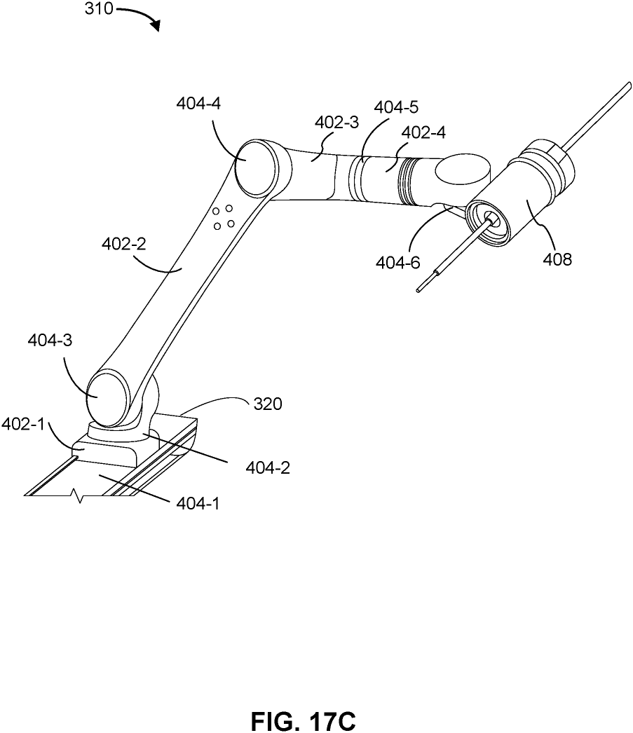

In some embodiments, the adjustable arm supports 320 can be configured to provide a base position for one or more of the robotic arms 310 for a robotic medical procedure. A robotic arm 310 can be positioned relative to the patient support platform 302 by translating the robotic arm 310 along a length of its underlying arm support 320 and/or by adjusting a position and/or orientation of the robotic arm 310 via one or more joints and/or links (see, e.g., FIG. 17A). In some embodiments, the arm support 320 pose can be changed via manual manipulation, teleoperation, and/or power assisted motion.

In some embodiments, the adjustable arm support 320 can be translated along a length of the patient support platform 302. In some embodiments, translation of the arm support 320 along a length of the patient support platform 302 causes one or more of the robotic arms 310 supported by the arm support 320 to be simultaneously translated with the arm support or relative to the arm support. In some embodiments, the arm support 320 is capable of being translated while keeping one or more of the robotic arms stationary with respect to the base 306 of the robotic medical system 300.

In the example of FIG. 15, the adjustable arm support 320 is located along a length of the patient support platform 302. In some embodiments, the adjustable arm support 320 extends across a partial or full length of the patient support platform 302, and/or across a partial or full width of the patient support platform 302.

During a robotic medical procedure, one or more of the robotic arms 310 can also be configured to hold instruments 312 (e.g., robotically controlled medical instruments or tools, such as an endoscope and/or any other instruments (e.g., sensors, illumination instrument, cutting instrument, etc.) that may be used during surgery), and/or be coupled to one or more accessories, including one or more cannulas, in accordance with some embodiments.

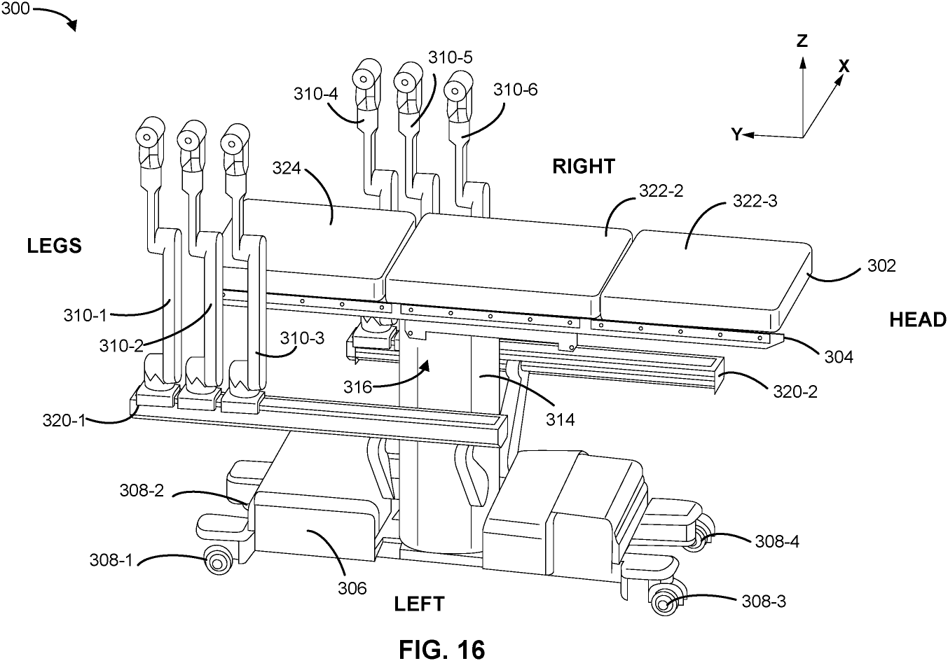

FIG. 16 illustrates another view of the robotic medical system 300 in FIG. 15 according to some embodiments. In this example, the robotic medical system 300 includes six robotic arms 310-1, 310-2, 310-3, 310-4, 310-5, and 310-6. The patient platform 302 is supported by a column 314 that extends between the base 306 and the patient platform 302. In some embodiments, the patient platform 302 includes a tilt mechanism 316. The tilt mechanism 316 can be positioned between the column 314 and the patient platform 302 to allow the patient platform 302 to pivot, rotate, or tilt relative to the column 314. The tilt mechanism 316 can be configured to allow for lateral and/or longitudinal tilt of the patient platform 302. In some embodiments, the tilt mechanism 316 allows for simultaneous lateral and longitudinal tilt of the patient platform 302.

FIG. 16 shows the patient platform 302 in an untilted state or position. In some embodiments, the untilted state or position is a default position of the patient platform 302. In some embodiments, the default position of the patient platform 302 is a substantially horizontal position as shown in FIG. 16. As illustrated, in the untilted state, the patient platform 302 can be positioned horizontally or parallel to a surface that supports the robotic medical system 300 (e.g., the ground or floor). In some embodiments, the term “untilted” refers to a state in which the angle between the default position and the current position is less than a threshold angle (e.g., less than 5 degrees, or less than an angle that would cause the patient to shift on the patient platform, etc.). In some embodiments, the term “untilted” refers to a state in which the patient platform is substantially perpendicular to the direction of gravity, irrespective of the angle formed by the surface that supports the robotic medical system relative to gravity.

With continued reference to FIG. 16, in the illustrated example of the robotic medical system 300, the patient platform 302 includes a support 304. In some embodiments, the support 304 includes a rigid support structure or frame, and can support one or more surfaces, pads, or cushions 322. An upper surface of the patient platform 302 can include a support surface 324. During a medical procedure, a patient can be placed on the support surface 324.

FIG. 16 shows the robotic arms 310 and the adjustable arm supports 320 in an example deployed configuration in which the robotic arms 310 reach above the patient platform 302. In some embodiments, due to the configuration of the robotic medical system 300, which enables stowage of different components beneath the patient platform 302, the robotic arms 310 and the arm supports 320 can occupy a space underneath the patient platform 302. Thus, in some embodiments, the tilt mechanism 316 has a low-profile and/or low volume in order to increase the space available for storage below.

FIG. 16 also illustrates an example, x, y, and z coordinate system that may be used to describe certain features of the embodiments disclosed herein. It will be appreciated that this coordinate system is provided for purposes of example and explanation only and that other coordinate systems may be used. In the illustrated example, the x-direction or x-axis extends in a lateral direction across the patient platform 302 when the patient platform 302 is in an untilted state. In some configurations, the x-direction extends across the patient platform 302 from one lateral side (e.g., the right side) to the other lateral side (e.g., the left side) when the patient platform 302 is in an untilted state. The y-direction or y-axis extends in a longitudinal direction along the patient platform 302 when the patient platform 302 is in an untilted state. That is, the y-direction extends along the patient platform 302 from one longitudinal end (e.g., the head end) to the other longitudinal end (e.g., the legs end) when the patient platform 302 is in an untilted state. In an untilted state, the patient platform 302 can lie in or be parallel to the x-y plane, which can be parallel to the floor or ground. In the illustrated example, the z-direction or z-axis extends along the column 314 in a vertical direction. In some embodiments, the tilt mechanism 316 is configured to laterally tilt the patient platform 302 by rotating the patient platform 302 about a lateral tilt axis that is parallel to the y-axis. The tilt mechanism 316 can further be configured to longitudinally tilt the patient platform 302 by rotating the patient platform 302 about a longitudinal tilt axis that is parallel to the x-axis.

### A. Robotic Arms.

FIGS. 17A-17C illustrate different views of a robotic arm 210 according to some embodiments. FIG. 17A illustrates that the robotic arm 310 includes a plurality of links 402 (e.g., linkages). The links 402 are connected by one or more joints 404. Each of the joints 404 includes one or more degrees of freedom (DoFs). In FIG. 17A, the joints 404 include a first joint 404-1 (e.g., a base joint or an A0 joint) that is located at or near a base 406 of the robotic arm 310. In some embodiments, the base joint 404-1 includes a prismatic joint that allows the robotic arm 310 to translate along the arm support 320 (e.g., along the y-axis). The joints 404 also include a second joint 404-2. In some embodiments, the second joint 404-2 rotates with respect to the base joint 404-1. The joints 404 also include a third joint 404-3 that is connected to one end of link 402-2. In some embodiments, the joint 404-3 includes multiple DoFs and facilitates both tilt and rotation of the link 402-2 tilt with respect to the joint 404-3.

FIG. 17A also shows a fourth joint 404-4 that is connected to the other end of the link 402-2. In some embodiments, the joint 404-4 includes an elbow joint that connects the link 402-2 and the link 402-3. The joints 404 further include a pair of joints 404-5 (e.g., a wrist roll joint) and 404-6 (e.g., a wrist pitch joint), which is located on a distal portion of the robotic arm 310. A proximal end of the robotic arm 310 may be connected to a base 406 and a distal end of the robotic arm 310 may be connected to an advanced device manipulator (ADM) 408 (e.g., a tool driver, an instrument driver, or a robotic end effector, etc.). The ADM 408 may be configured to control the positioning and manipulation of a medical instrument 312 (e.g., a tool, a scope, etc.).

The robotic arm 310 can also include a cannula sensor 410 for detecting presence or proximity of a cannula to the robotic arm 310. In some embodiments, the robotic arm 310 is placed in a docked state (e.g., docked position) when the cannula sensor 410 detects presence of a cannula (e.g., via one or more processors of the robotic medical system 300). In some embodiments, when the robotic arm 310 is in a docked position, the robotic arm 310 can execute null space motion to maintain a position and/or orientation of the cannula, as discussed in further detail below. Conversely, when no cannula is detected by the cannula sensor 410, the robotic arm 310 is placed in an undocked state (e.g., undocked position).

In some embodiments, and as illustrated in FIG. 17A, the robotic arm 310 includes an input or button 412 (e.g., a donut-shaped button, or other types of controls, etc.) that can be used to place the robotic arm 310 in an admittance mode (e.g., by depressing the button 412). The admittance mode is also referred to as an admittance scheme or admittance control. In the admittance mode, the robotic system 310 measures forces and/or torques (e.g., imparted on the robotic arm 310) and outputs corresponding velocities and/or positions. In some embodiments, the robotic arm 310 can be manually manipulated by a user (e.g., during a set-up procedure, or in between procedures, etc.) in the admittance mode. In some instances, by using admittance control, the operator need not overcome all of the inertia in the robotic medical system 300 to move the robotic arm 310. For example, under admittance control, when the operator imparts a force on the arm, the robotic medical system 300 can measure the force and assist the operator in moving the robotic arm 310 by driving one or more motors associated with the robotic arm 310, thereby resulting in desired velocities and/or positions of the robotic arm 310.

In some embodiments, the links 402 are detachably coupled to the medical instrument 312 (e.g., to facilitate ease of mounting and dismounting of the medical instrument 312 from the robotic arm 310). The joints 404 provide the robotic arm 310 with a plurality of degrees of freedom (DoFs) that facilitate control of the medical instrument 312 via the ADM 408. In embodiments including multiple robotic arms, each robotic arm can hold its own respective medical tool and pivot the medical tool about a remote center of motion.

FIG. 17B illustrates a front view of the robotic arm 310. FIG. 17C illustrates a perspective view of the robotic arm 310. In some embodiments, the robotic arm 310 includes a second input or button 414 (e.g., a push button) that is distinct from the button 412 in FIG. 17A, for placing the robotic arm 310 in an impedance mode (e.g., by a single press or continuous press and hold of the button 414). In this example, the button 414 is located between the joint 404-5 and the joint 404-6. The impedance mode is also referred to as impedance scheme or impedance control. In the impedance mode, the robotic medical system 300 measures displacements (e.g., changes in position and velocity) and outputs forces and/or torques to facilitate manual movement of the robotic arm. In some embodiments, the robotic arm 310 can be manually manipulated by a user (e.g., during a set-up procedure) in the impedance mode. In some embodiments, under the impedance mode, the operator’s movement of one part of a robotic arm 310 causes motion in one or more joints and/or links throughout the robotic arm 310.

In some embodiments, for admittance control, a force sensor or load cell can measure the force that the operator is applying to the robotic arm 310 and move the robotic arm 310 in a way that feels light. Admittance control may feel lighter than impedance control because, under admittance control, one can hide the perceived inertia of the robotic arm 310 because motors in the controller can help to accelerate the mass. In contrast, with impedance control, the user is responsible for most if not all mass acceleration, in accordance with some embodiments.

In some circumstances, depending on the position of the robotic arm 310 relative to the operator, it may be inconvenient to reach the button 412 and/or the button 414 to activate a manual manipulating mode (e.g., the admittance mode and/or the impedance mode). Accordingly, under these circumstances, it may be convenient for the operator to trigger the manual manipulation mode other than by buttons.

In some embodiments, the robotic arm 310 includes a single button (e.g., the button 412 or 414) that can be used to place the robotic arm 310 in the admittance mode and/or the impedance mode (e.g., by using different presses, such as a long press, a short press, press and hold etc.). In some embodiments, the robotic arm 310 is placed in impedance mode by a user pushing on arm linkages (e.g., the links 402) and/or joints (e.g., the joints 404) and overcoming a force threshold. In some embodiments, the admittance mode and the impedance mode are common in that they both allow the user to grab the robotic arm 310 and command motion by directly interfacing with it.

In some embodiments, the robotic arm 310 includes an input control for activating an arm follow mode. For example, in some embodiments, the robotic arm 310 includes a designate touch point that is located on a link 402 or a joint 404 of the robotic arm (e.g., an outer shell of the link 402 or a button 416). User interaction (e.g., user touch, contact, etc.) with the designate touch point activates the arm follow mode. In some embodiments, the robotic arm 310 includes multiple touch points. User interaction with any (e.g., one or more) of the touch points activates the arm follow mode.

During a medical procedure, it can be desirable to have the ADM 408 of the robotic arm 310 and/or a remote center of motion (RCM) of the instrument 312 coupled thereto kept in a static pose (e.g., position and/or orientation). An RCM may refer to a point in space where a cannula or other access port through which a medical instrument 312 is inserted is constrained in motion. In some embodiments, the medical instrument 312 includes an end effector that is inserted through an incision or natural orifice of a patient while maintaining the RCM. In some embodiments, the medical instrument 312 includes an end effector that is in a retracted state during a setup process of the robotic medical system.

In some circumstances, the robotic medical system 300 can be configured to move one or more links 402 of the robotic arm 310 within a “null space” to avoid collisions with nearby objects (e.g., other robotic arms), while the ADM 408 of the robotic arm 310 and/or the RCM are maintained in their respective poses (e.g., positions and/or orientations). The null space can be viewed as the set of joint states through which a robotic arm 310 can move that does not result in movement of the ADM 408 and/or RCM, thereby maintaining the position and/or the orientation of the medical instrument 312 (e.g., within a patient). In some embodiments, a robotic arm 310 can have multiple positions and/or configurations available for each pose of the ADM 408.

For a robotic arm 310 to move an instrument to a desired pose in space, in certain embodiments, the robotic arm 310 has at least six DoFs - three DoFs for translation (e.g., X, Y, and Z positions) and three DoFs for rotation (e.g., yaw, pitch, and roll). In some embodiments, each joint 404 provides the robotic arm 310 with a single DoF, and thus, the robotic arm 310 has at least six joints to achieve freedom of motion to position the ADM 408 at any pose in space. To further maintain the ADM 408 of the robotic arm 310 and/or the remote center or motion in a desired pose, the robotic arm 310 may further have at least one additional “redundant j oint.” Thus, in certain embodiments, the system includes a robotic arm 310 having at least seven joints 404, providing the robotic arm 310 with at least seven DoFs. In some embodiments, the robotic arm 310 includes a subset of joints 404 each having more than one degree of freedom thereby achieving the additional DoFs for null space motion. However, depending on the embodiment, the robotic arm 310 may have a greater or fewer number of DoFs.

Furthermore, as described with respect to FIG. 7, the arm support 320 (e.g., adjustable arm support) can provide several degrees of freedom, including lift, lateral translation, tilt, etc. Thus, depending on the embodiment, a robotic medical system can have many more robotically controlled degrees of freedom beyond just those in the robotic arms 310 to provide for null space movement and collision avoidance. In a respective embodiment of these embodiments, the end effectors of one or more robotic arms (and any tools or instruments coupled thereto) and a remote center along the axis of the tool can advantageously maintain in pose and/or position within a patient.

A robotic arm 310 having at least one redundant DoF has at least one more DoF than the minimum number of DoFs for performing a given task. For example, a robotic arm 310 can have at least seven DoFs, where one of the joints 404 of the robotic arm 310 can be considered a redundant joint, in accordance with some embodiments. The one or more redundant joints can allow the robotic arm 310 to move in a null space to both maintain the pose of the ADM 408 and a position of an RCM and avoid collision(s) with other robotic arms or objects.

In some embodiments, the robotic medical system 300 is configured to perform collision avoidance to avoid collision(s), e.g., between adjacent robotic arms 310, by taking advantage of the movement of one or more redundant joints in a null space. For example, when a robotic arm 310 collides with or approaches (e.g., within a defined distance of) another robotic arm 310, one or more processors of the robotic medical system 300 can be configured to detect the collision or impending collision (e.g., via kinematics). Accordingly, the robotic medical system 300 can control one or both of the robotic arms 310 to adjust their respective joints within the null space to avoid the collision or impending collision. In an embodiment including at least a pair of robotic arms, a base of one of the robotic arms and its end effector can stay in its pose, while links or joints therebetween move in a null space to avoid collisions with an adjacent robotic arm.

### B. Example Port Placements for Different Surgical Procedures.

As described earlier, the robotic arm poses and port placement relative to target anatomy is vital to a successful medical procedure. Medical procedures typically require precise port locations and have a small tolerance for errors.

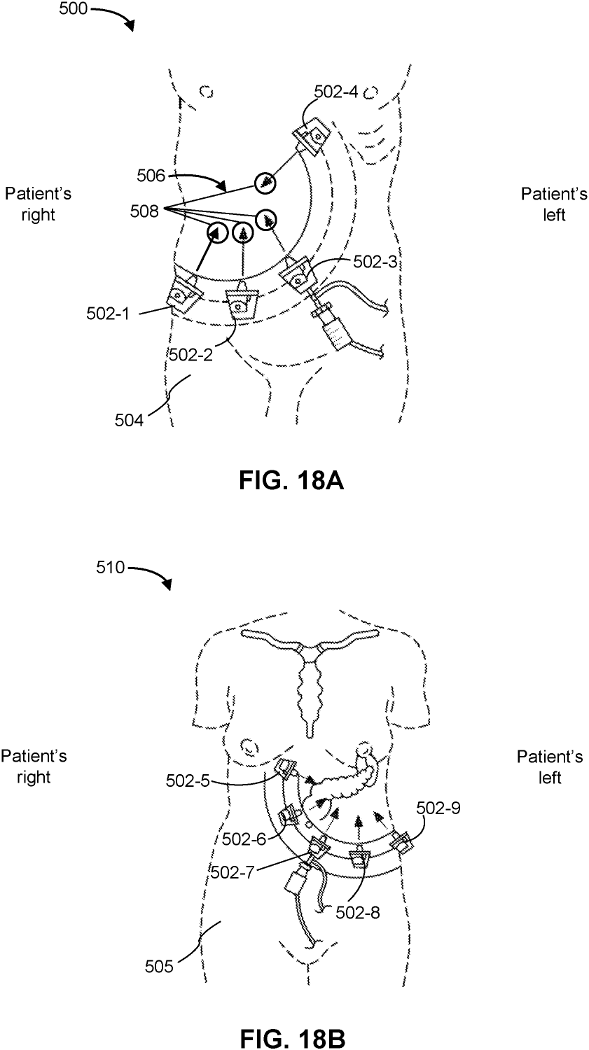

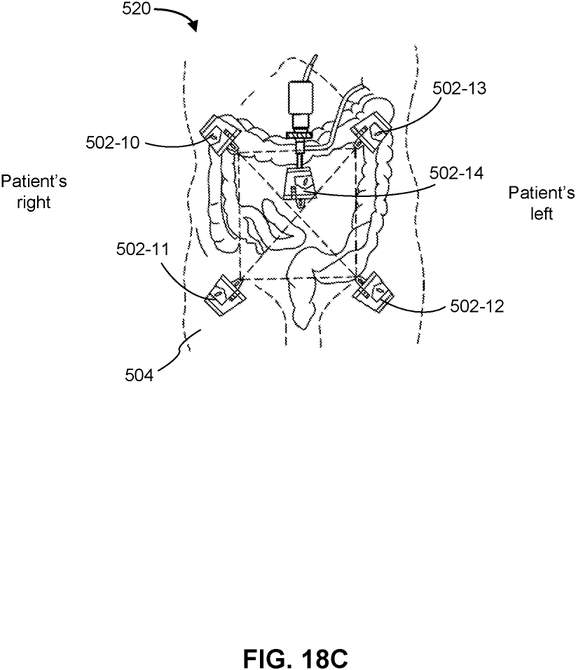

FIGS. 18A-18C illustrate example cannula placement for different surgical procedures in accordance with some embodiments. FIG. 18A illustrates an example surgical environment 500 that includes placement of four cannulas 502-1, 502-2, 502-3, and 502-4 at a number of anatomical locations within a patient 504, to provide access to at least a portion of the patient’s anatomy 506 (e.g., abdomen). The cannulas 502 are capable of passing through port locations 508 in the patient (or in the patient’s anatomy 506). As used herein, a port location (e.g., a port, a port of entry, an entry point, a port region, a port area, or a port position, etc.) refers to a position on a patient’s body through which a medical tool/instrument (e.g., held by a robotic arm) can be inserted and constrained in motion. In some embodiments, the port location corresponds to an incision point (or an incision region) that is made through the skin of the patient to facilitate a medical operation or procedure. In some embodiments, the port location corresponds to a natural orifice, such as a mouth of the patient (e.g., for a bronchoscopy procedure). In the example of FIG. 18A, the illustrated placement of the cannulas 502 may allow access to the right upper quadrant of the patient’s abdomen.

In some embodiments, a camera is docked to one of the cannulas 502 (e.g., cannula 502-3) to provide a view of the patient’s anatomy (e.g., a right upper quadrant in FIG. 18A). Medical instruments may be docked to one or more of the cannulas 502 (e.g., 502-1, 502-2, and/or 502-4). Each of the cannulas 502 provides access to the patient’s anatomy 506 from a unique location and/or orientation, allowing for flexibility in the manner in which the medical procedure is performed. The angles from which medical tools may access the patient’s anatomy 506 from the cannulas 502 are illustrated by arrows extending from the respective cannulas 502. As an example, during an initial stage of a medical procedure, a physician may use medical tools docked to the cannulas 502-1 and 502-2, and at a subsequent stage may use medical tools docked to the cannulas 502-3 and 502-4. By using medical tools docked to different cannulas 502, the physician may be able to access the patient’s anatomy 506 from different angles, allowing more options for direct access to various portions of the patient’s anatomy 506.

In some embodiments, as illustrated in FIG. 18A, the cannulas 502-1 and 502-4 provide access to the patient’s anatomy 506 from substantially opposite sides, allowing the physician access to both sides of the anatomy 506 by simply selecting the medical tools docked to the cannulas 502-1 and 502-4.

FIG. 18B illustrates an example surgical environment 510 that includes five cannulas 502-5 to 502-9 placed in anatomical locations of the patient 505, to provide access to a left upper quadrant of the patient’s abdomen.

FIG. 18C illustrates an example surgical environment 520 that includes five cannulas 502-10 to 502-14 in anatomical locations of a patient 504, to provide access to multiple quadrants of an abdomen of the patient 504. The placement of the cannulas 502 in FIG. 18C may be provided to perform a colectomy as an example surgical procedure.

### C. Example Workflows for Optimal Robotic Arm Poses

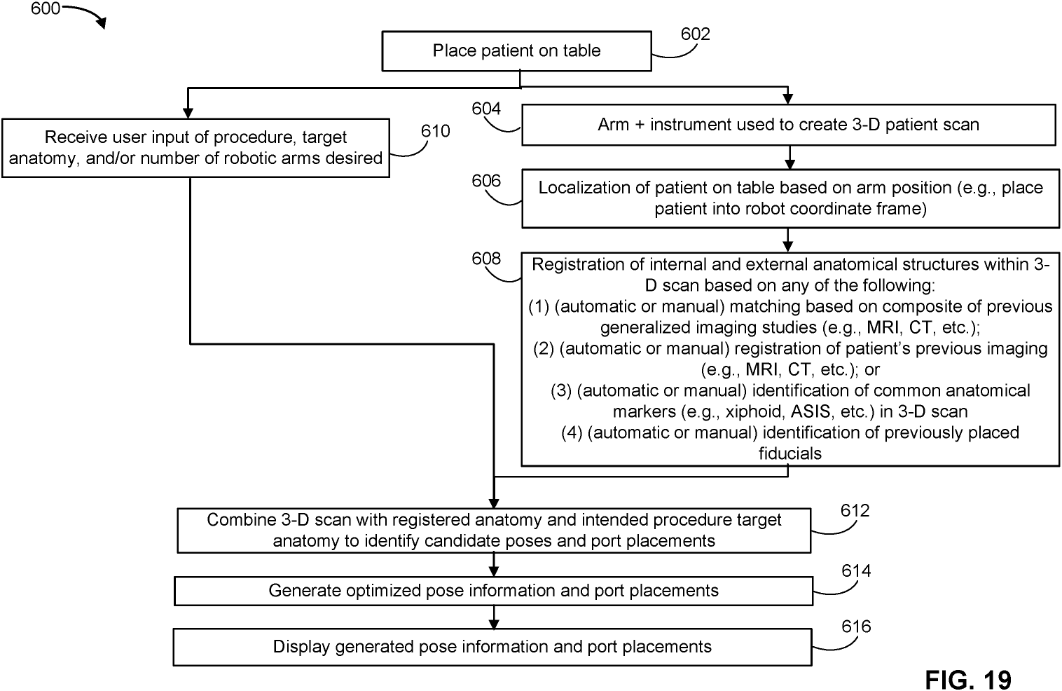

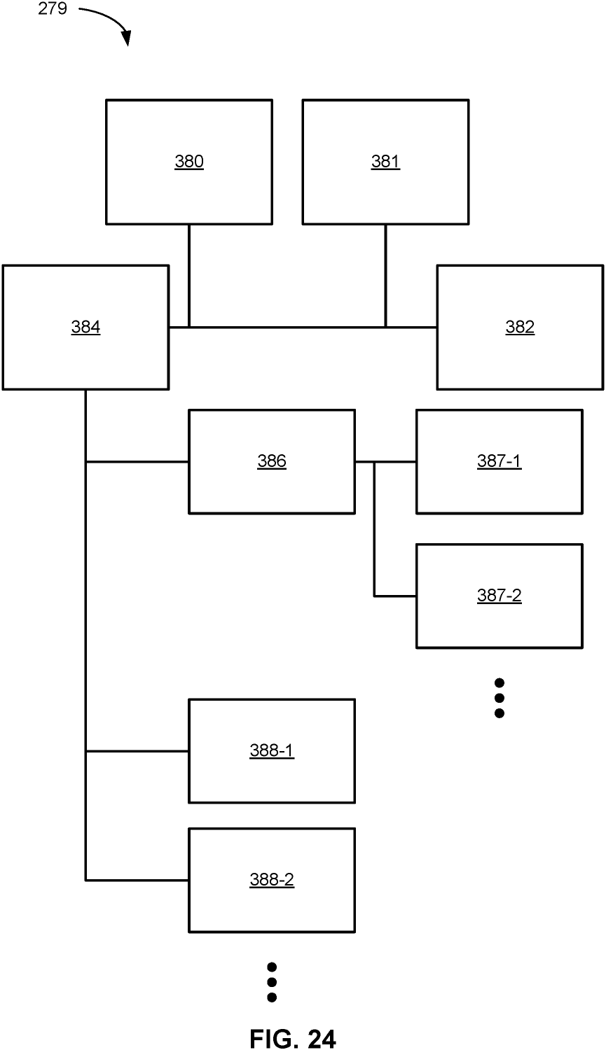

FIG. 19 illustrates an example workflow 600 for generating optimal port locations and robot arm poses for a medical operation or procedure, in accordance with some embodiments. In some embodiments, the workflow 600 is implemented as a set of instructions that are executed by one or more processors of a robotic medical system (e.g., processors 380, FIG. 24), for recommending port placements and starting poses to avoid collisions (e.g., between a patient and a robotic arm, between two or more robotic arms, between a robotic arm and a surgical tool, between two or more surgical tools, etc.) during surgery.

The workflow 600 includes placing (602) the patient on a table (e.g., patient support platform 302, a bed, etc.) of the robotic medical system in accordance with some embodiments. In some embodiments, the patient is arranged on the table in a position that they will be in during the medical operation or procedure.

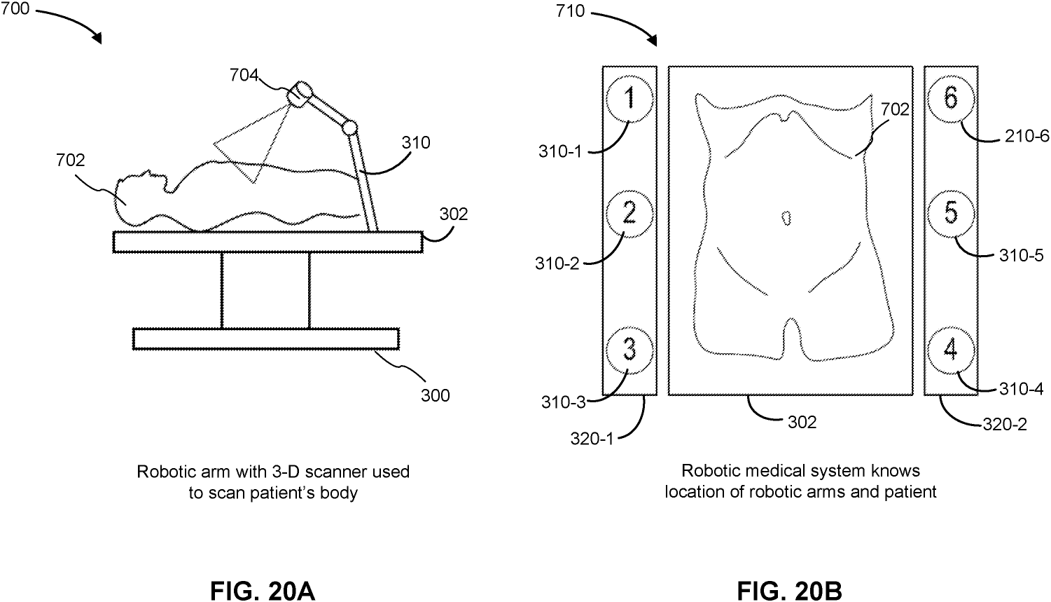

The workflow 600 includes obtaining (604) (e.g., creating, generating, receiving, determining, collecting, etc.) a 3-D scan (e.g., 3-D data, imaging data, scan data, etc.) that includes a view of a patient in accordance with some embodiments. FIGS. 20A and 20B illustrate example 3-D scanning and registration processes of a patient relative to a robotic medical system, in accordance with some embodiments. FIG. 20A shows a side view 700 that illustrates a patient 702 positioned on a patient support platform 302 of a robotic medical system 300. In this example, a 3-D scanner 704 is attached to a robotic arm 310 of the robotic medical system 300. FIG. 20B illustrates a top view 710 that includes a portion (e.g., an anatomy, such as an abdomen) of the patient 702 and positions of robotic arms 310-1 to 310-6 and adjustable arm supports 320-1 and 320-2 of the robotic medical system 300.

In some embodiments, the 3-D scanner 704 is included with (e.g., is a component of, forms a part of, etc.) the robotic medical system 300. For example, the 3-D scanner 704 can be a component of the robotic medical system 300 that is either integrated into or attached onto the robotic medical system 300. For example, the 3-D scanner 704 can be located (e.g., positioned) on a robotic arm 310, a patient support platform 302 (e.g., bed), an adjustable arm support 320, a tower pendant, and/or any portion of the robotic medical system 300 that includes a view of the patient.

In some embodiments, the 3-D scanner 704 is part of an instrument (e.g., a medical tool such as a laparoscope) that is held by a robotic arm 310 of the robotic medical system 300. In some embodiments, the 3-D scanner 704 is a separate accessory (e.g., a standalone scanner, a handheld scanner etc.) that is communicatively connected with the robotic medical system 300, and attached to (e.g., mounted on) a robotic arm 310, a patient support platform 302 (e.g., bed), an adjustable arm support 320, a tower pendant, and/or any portion of the robotic medical system 300 that includes a view of the patient. In some embodiments, the 3-D scanner 704 is mounted on a wall and/or ceiling of an operating room in which the robotic medical system 300 is located.

In some embodiments, the 3-D scanner 704 uses time-of-flight (e.g., laser imaging, detection, and ranging (LIDAR)), dot pattern recognition, stereo photography (e.g., using a stereo camera), and/or other established scan technologies to perform the 3-D scanning. It should be noted that these scan technologies are examples and not intended to be limiting.

The 3-D scanner 704 creates (e.g., generates, obtains, etc.) data (e.g., scan data, 3-D scan data, etc.) that includes a view of the patient 702. In some embodiments, the 3-D scan data includes visual data (e.g., imaging data) of at least a portion of the patient 702 and an environment (e.g., spaces and/or other objects) surrounding the patient 702. The scan data can capture size (e.g., dimensions), shape, and/or depth information of at least a portion of the patient 702 and one or more objects surrounding the patient 702 (e.g., a patient support platform, a medical tool, etc.). In some embodiments, the scan data includes dimensional information (e.g., information such as distance, angle, etc.) that can be used to determine relative separations (e.g., spatial separation, angular separation) between the patient 702 and other objects.

The workflow 600 includes localization (606) of the patient on the table (e.g., patient support platform 302) based on the position of a robotic arm 310 in accordance with some embodiments. For example, the 3-D scan data can include a view of the patient and a part of the robotic medical system, such as a robotic arm, the patient support platform, and/or an adjustable arm support (or part thereof). Because the robotic medical system 300 can determine positions and/or orientations of the robotic arm, the patient support platform, and/or the adjustable arm support (e.g., through data obtained by sensors and/or encoders that are located throughout the robotic medical system), the robotic medical system 300 can localize (e.g., determine one or more positions of) the patient relative to a component of the robotic medical system 300 (e.g., relative to a robotic arm 310).

In some embodiments, after localizing the patient based on the position of a robotic arm 310, the robotic medical system proceeds to place the patient on a coordinate system of the robotic medical system 300, and determine coordinates corresponding to different parts of the patient (e.g., head, toes, belly button, etc.) using the coordinate system. For example, in some embodiments, the robotic medical system 300 includes a coordinate system (e.g., a robot coordinate system, a coordinate frame, a system frame, etc.) and respective positions of the patient support platform 302, the robotic arms 310, and/or the adjustable arm supports 320 can be represented as coordinates (e.g., x-, y-, and z-coordinates) on the coordinate system. The robotic medical system 300 can use the scan data (e.g., imaging data that includes a view of the patient and part of a robotic arm 310) obtained by the 3-D scanner 704, to “match” the patient frame with the system frame (e.g., robotic arm frame) and “register” the patient onto the coordinate system.

The workflow 600 includes registration (608) (e.g., determination) of anatomical structures (e.g., external and/or internal anatomical structures) within the 3-D scan by the robotic medical system 300 in accordance with some embodiments. In some embodiments, the robotic medical system 300 compares (e.g., matches) (e.g., automatically or manually based on a user input) the 3-D scan data with previous generalized imaging data (e.g., heuristics data, prior imaging data, imaging data from a database, etc.), such as MRI imaging data and/or CT scan data. The robotic medical system 300 identifies one or more anatomical structures in accordance with the comparison (e.g., the robotic medical system 300 identifies one or more anatomical structures in accordance with a determination that an anatomical structure shown in the 3-D scan data matches an anatomical structure in a database of imaging data).

In some embodiments, the robotic medical system 300 obtains pre-operative imaging data (e.g., MRI imaging data, CT scan data, etc.) corresponding to the patient. The robotic medical system 300 compares (e.g., automatically or manually based on user input, comparing side-by-side, overlaying the two sets of data, etc.) the 3-D scan data with the pre-operative imaging data, and identifies (e.g., registers) one or more anatomical structures in accordance with the comparison.

In some embodiments, the robotic medical system 300 registers (e.g., identifies, determines, etc.) (e.g., automatically or manually based on user input) one or more anatomical markers (e.g., anatomical landmarks, anatomical structures) of the patient from the 3-D scan data. For example, the one or more anatomical markers can include an external anatomical marker such as the belly button, or an internal anatomical marker (e.g., the xiphoid process or the anterior superior iliac spine (ASIS)) that can be inferred based on the 3-D scan data. In some configurations, the robotic medical system 300 utilizes image processing software or pattern recognition software to identify positions of one or more external anatomical markers. In some configurations, the robotic medical system 300 determines positions of one or more internal anatomical markers from the positions of the one or more external anatomical markers (e.g., an internal anatomical marker is located below a position having certain distances to two or more external anatomical markers).

In some embodiments, the robotic medical system registers (e.g., automatically or manually based on user input) one or more anatomical markers based on fiducials (e.g., anatomical side markers) that are placed on the patient prior to collecting the 3-D scan. In some embodiments, positions of the one or more fiducials are deemed to correspond to positions of the one or more anatomical markers. In some embodiments, positions of the one or more anatomical markers are determined based on positions of the one or more fiducials. For example, positions of the one or more anatomical markers are deemed to correspond to a predefined distance below the one or more fiducials. In another example, a position of an anatomical marker is deemed to correspond to a position determined based on two or more fiducials (e.g., having an equal distance to the two or more fiducials).

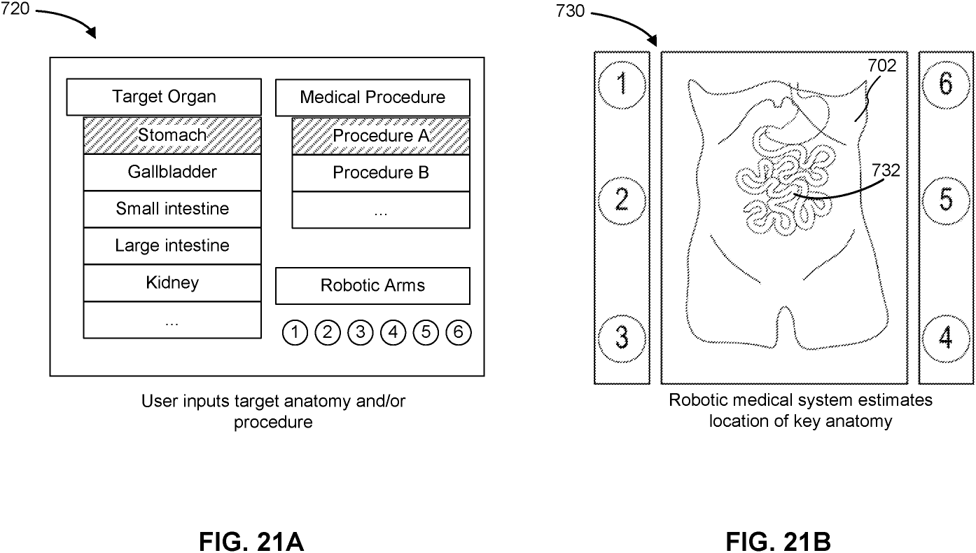

The workflow 600 includes receiving (610) user input from a user of the robotic medical system 300, such as a surgeon, a nurse, or a surgeon assistant, in accordance with some embodiments. The input can include identification of a medical procedure to be performed on the patient, a target anatomy (or target anatomies), and/or a number of robotic arms (and/or a selection of robotic arms) to be used for the procedure. FIG. 21A illustrates user input of a target anatomy or procedure in accordance with some embodiments. As shown in FIG. 21A, the operator may provide the user input through a user interface. In some embodiments, the robotic medical system includes a touch-sensitive display (e.g., a touch screen) and the operator may provide the user input through the touch-sensitive display. Alternatively, the operator may provide the user input through any other input devices (e.g., a mouse, a keyboard, one or more buttons or switches, etc.). FIG. 21B illustrates the robotic medical system 300 estimating a location of a target anatomy (e.g., stomach). In some embodiments, the robotic medical system 300 estimates a location of the target anatomy based on the approaches described with respect to operation 608 of the workflow 600, and/or in combination with the user input in operation 610.

The workflow 600 includes combining (612) the 3-D scan data (e.g., operation 604) with registered anatomy (e.g., obtained from operation 608) and the intended (or selected) procedure and/or target anatomy (e.g., obtained from operation 610) to identify candidate patient-specific robotic arm poses and port placements for the medical procedure in accordance with some embodiments. In some embodiments, the combining includes simulating the medical procedure based on the combined data (e.g., mapping robotic arm trajectories during the medical procedure).

The workflow 600 includes generating (614) one or more recommended (e.g., optimized) port locations and robotic arm poses that are specific to the patient and the procedure (medical operation). For example, the robotic medical system identifies a plurality of locations that are within a first predefined distance from the target anatomy (e.g., the target organ), excludes locations that are adjacent to one or more exclusion regions (also called “keep out zones”, such as regions where other critical anatomy, such as organs, are located, regions where other tools or other accessories are located, identifies a number of ports associated with the intended (or selected) procedure and orientations of the ports relative to the target anatomy (e.g., from memory of the robotic medical system storing the number and orientations of the ports), selects locations that are within the predefined distance from the target anatomy in the identified orientations. Furthermore, the robotic medical system identifies starting poses for the robotic arms involved in the procedure. The robotic medical system simulates the procedure and movement of the robotic arms during the procedure to identify any collisions that may occur (e.g., between robotic arms and/or between a robotic arm and the patient, other equipment, or persons).

In some embodiments, the robotic medical system identifies one or more potential starting poses (e.g., each corresponding to a recommended set of port placements) and simulates the procedure and movements for each potential starting pose. For example, the robotic medical system may select a plurality of potential starting poses from a set of predefined potential starting poses. In another example, the robotic medical system may generate a plurality of potential starting poses by varying a joint angle for one or more joints (e.g., generating the plurality of potential starting poses corresponding to particular joint angles for a particular joint). In some embodiments, the robotic medical system selects the potential starting pose that results in a minimum number of collisions (e.g., zero) as the recommended starting pose. For example, the robotic medical system may record a number of collisions for a respective starting pose of the plurality of potential starting poses (e.g., based on the simulation) and select a starting pose with a least number of collisions among the plurality of potential starting poses. In another example, the robotic medical system may select a starting pose that has less than a predefined number of collisions. In some embodiments, the port locations that are separated by at least a second predefined distance are selected (e.g., to provide a sufficient distance between the tools). In some embodiments, a value function based on each port location’s distance to the target anatomy, each port location’s distances to other port locations, each port location’s distances to one or more exclusion regions, and/or an orientation to the target anatomy provided by each port location is used (in an optimization process) for optimizing the port locations.

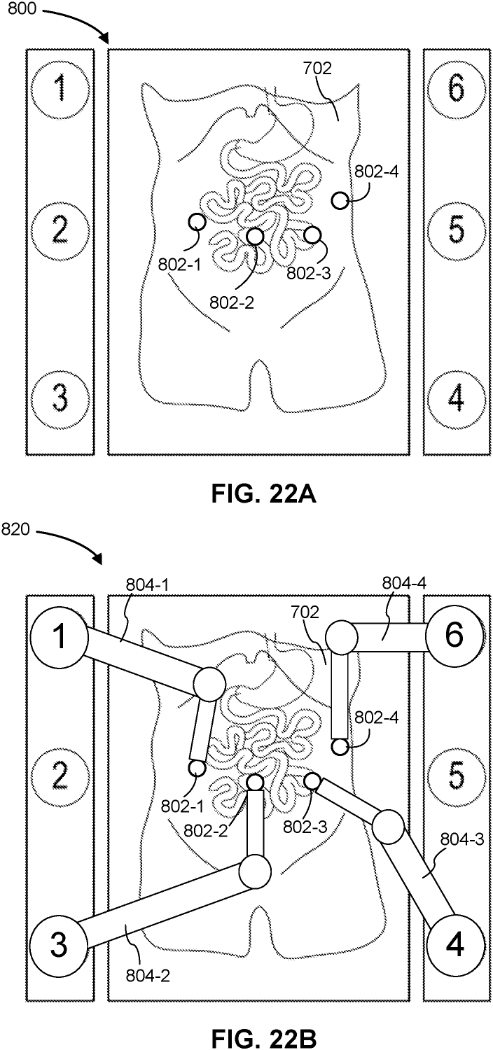

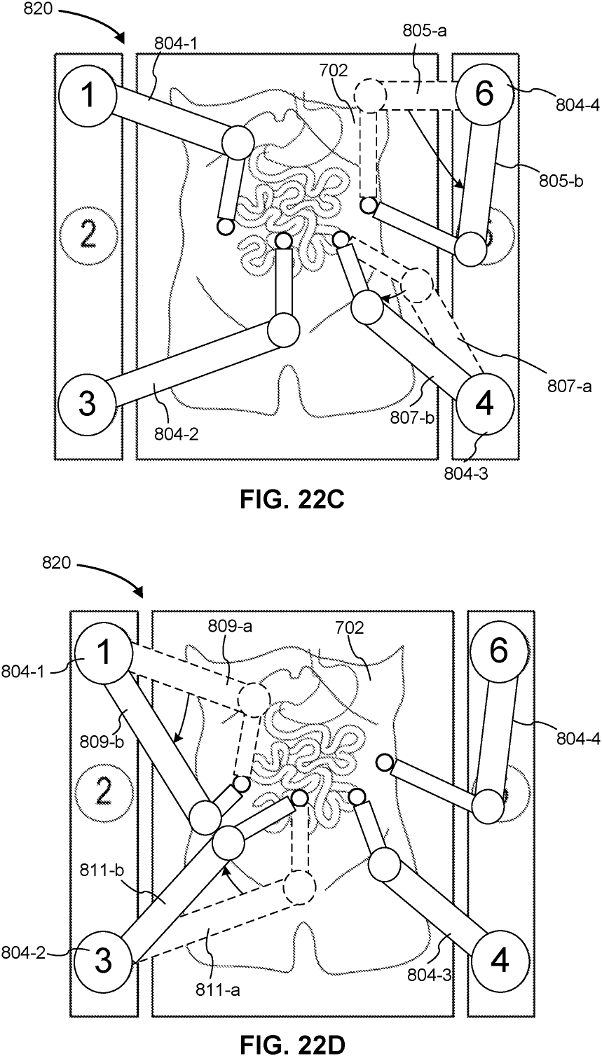

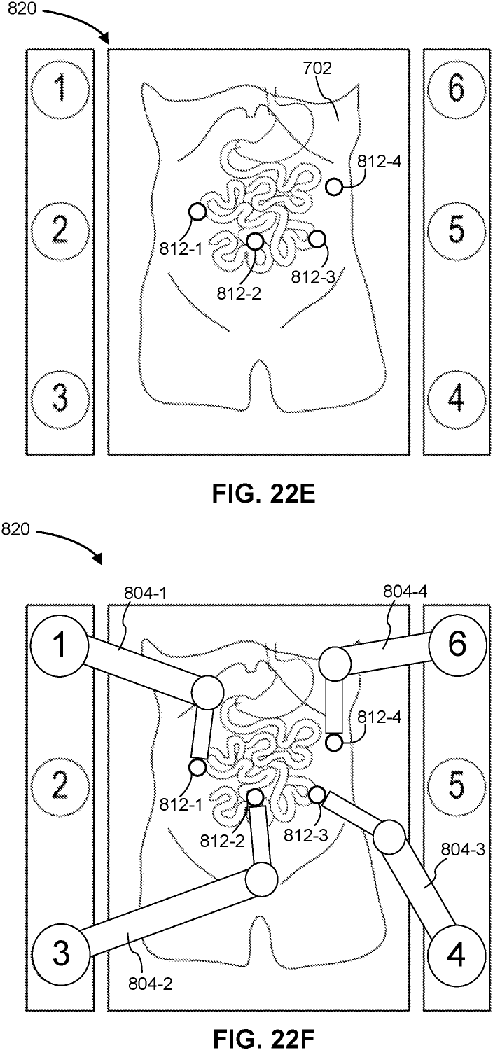

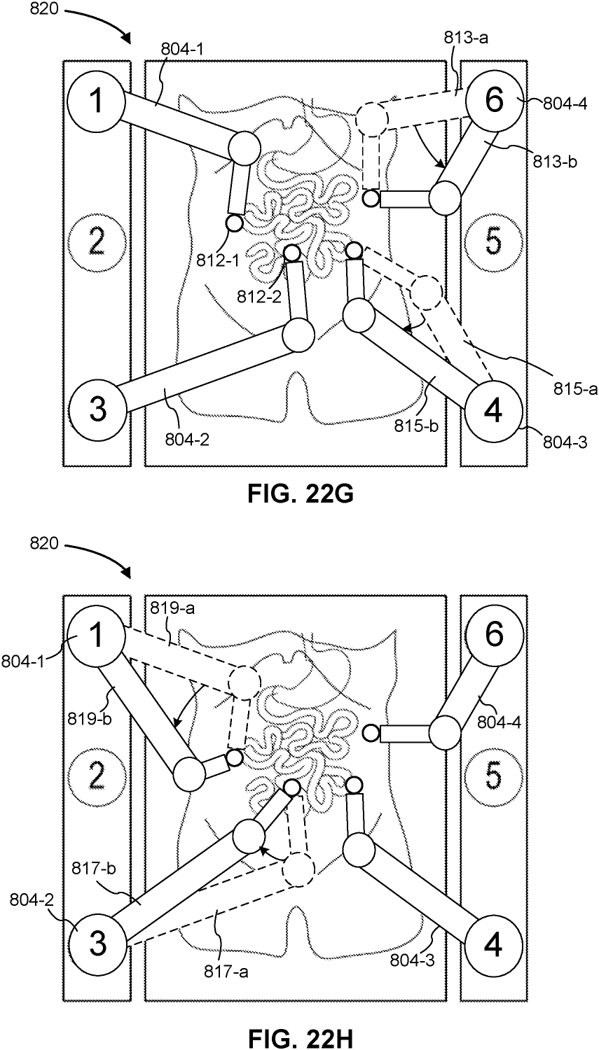

The workflow 600 includes displaying (616) (or causing to be displayed) the optimized port locations and robotic arm poses in accordance with some embodiments. FIG. 22A illustrates a top view 800 of a patient 702 that includes optimal port locations 802 displayed (e.g., projected) on the belly of the patient 702. FIG. 22B illustrates the optimized port locations and robotic arm poses displayed on a user interface 820 of the robotic medical system 300. In some embodiments, the user interface 820 is located on a surgeon viewer (e.g., a tower viewer, a tower pendant, etc.), or any display device that is communicatively connected with the robotic medical system 300. In some embodiments, the robotic medical system 300 displays the recommended arm poses and port locations using augmented-reality (AR) glasses that are communicatively connected with the robotic medical system 300. For example, the AR glasses can be worn by a user of the robotic medical system 300 during a setup phase for the medical procedure.

In some embodiments, the robotic medical system 300 causes the recommended robotic arm poses and port locations to be printed onto a medium. For example., in some embodiments, the recommended port locations are printed to a sterile, temporary tattoo that can be applied onto the patient. In some embodiments, the printed medium includes paper, film, and/or fabric that has been sterilized, on which the patient view, including the recommended port locations, is printed and attached (e.g., taped) onto the patient.

As mentioned above, FIG. 22A illustrates a set of recommended port locations 802 for a medical procedure, according to some embodiments. In the example of FIG. 22A the recommended port locations 802 include four port locations 802-1, 802-2, 802-3, and 802-4 in the area of the patient’s stomach. FIG. 22B illustrates an example set of initial (e.g., pre-docking) poses for the robotic arms 804 corresponding to the port locations 802. As shown in FIG. 22B, a subset of the total available robotic arms may be used for corresponding port locations (e.g., the robotic arms 804-1, 804-2, 804-3, and 804-4). For example, four robotic arms are used in a system that has up to six robotic arms. In some embodiments, each robotic arm has a plurality of joints (e.g., as discussed above with reference to FIGS. 17A-17C) and identifying an initial arm pose includes identifying an initial position for each joint of the plurality of joints.

FIGS. 22C and 22D illustrate example arm pose movements during a procedure, the example arm pose movements corresponding to the set of port locations 802 and the initial arm poses shown in FIG. 22B, according to some embodiments. FIG. 22C shows the user interface 820 with the robotic arm 804-4 moving from an initial position 805-a to a new position 805-b and the robotic arm 804-3 moving from an initial position 807-a to a new position 807-b. FIG. 22D shows the user interface 820 with the robotic arm 804-1 moving from an initial position 809-a to a new position 809-b and the robotic arm 804-2 moving from an initial position 811-a to a new position 811-b. FIG. 22D further shows a collision between the robotic arms 804-1 and 804-2 due to the movement. In accordance with some embodiments, the movements shown in FIGS. 22C and 22D correspond to a simulation of the movement of the robotic arms 804 during the medical procedure (e.g., a simulation performed by the system 279) based on the set of port locations 802 and the initial arm poses shown in FIG. 22B. In some embodiments, the robotic medical system forgoes selecting the port locations 802 and the initial poses shown in FIG. 22B as recommended port locations and recommended initial poses due to identifying the collision (e.g., unless no other combination of port locations and initial poses has fewer collisions).

FIG. 22E illustrates another set of port locations 812 for a medical procedure, according to some embodiments. In the example of FIG. 22E the recommended port locations 812 include four port locations 812-1, 812-2, 812-3, and 812-4 in the area of the patient’s stomach. In some embodiments, the port locations 812 correspond to a viable alternative to the port locations 802 for a given medical procedure. FIG. 22F illustrates an example set of initial (e.g., pre-docking) poses for the robotic arms 804 corresponding to the port locations 812.

FIGS. 22G and 22H illustrate example arm pose movements during a procedure, the example arm pose movements corresponding to the set of port locations 812 and the initial arm poses shown in FIG. 22F, according to some embodiments. FIG. 22G shows the user interface 820 with the robotic arm 804-4 moving from an initial position 813-a to a new position 813-b and the robotic arm 804-3 moving from an initial position 815-a to a new position 815-b. FIG. 22H shows the user interface 820 with the robotic arm 804-1 moving from an initial position 819-a to a new position 819-b and the robotic arm 804-2 moving from an initial position 817-a to a new position 817-b. In accordance with some embodiments, the movements shown in FIGS. 22G and 22H correspond to a simulation of the movement of the robotic arms 804 during the medical procedure (e.g., a simulation performed by the system 279) based on the set of port locations 812 and the initial arm poses shown in FIG. 22F. In accordance with some embodiments, the port locations 812 and initial poses shown in FIG. 22F may be selected as the recommended port locations and poses in accordance with the movement shown in FIGS. 22G and 22H resulting in no collisions (or less collisions than the alternative shown in FIGS. 22C and 22D).

Although not illustrated in FIGS. 22A-22H, in some embodiments, one or more additional robotic arms that do not correspond to a port placement are used during the procedure (e.g., to hold lights or equipment that does not require a corresponding port). In embodiments, where additional robotic arm(s) are present, the robotic medical system accounts for those arm(s) when determining initial poses and simulating collisions during the medical operation or procedure.

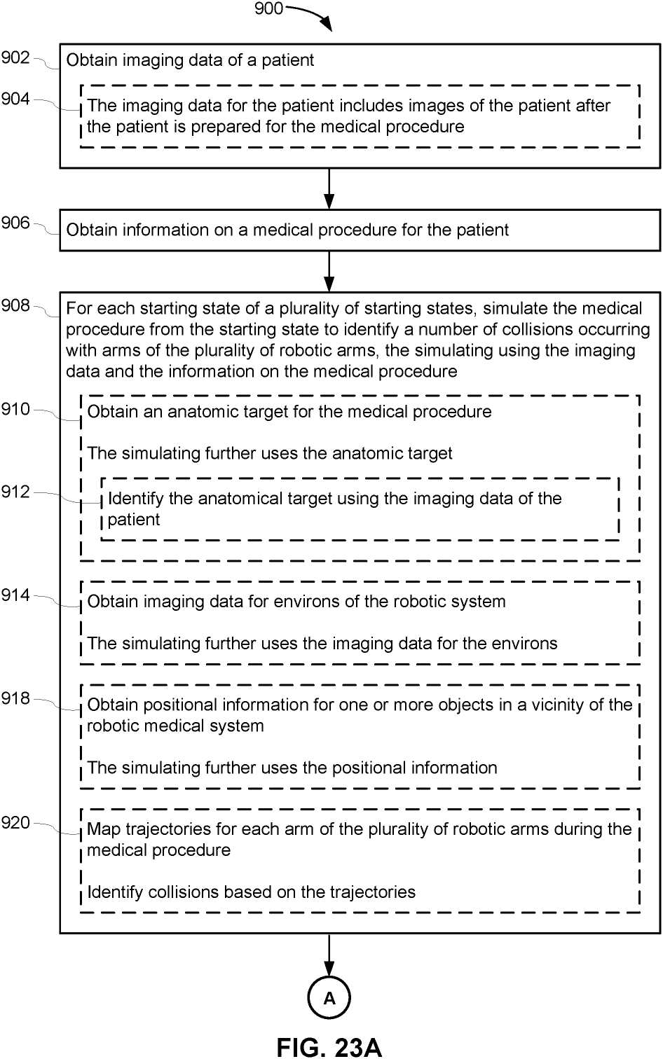

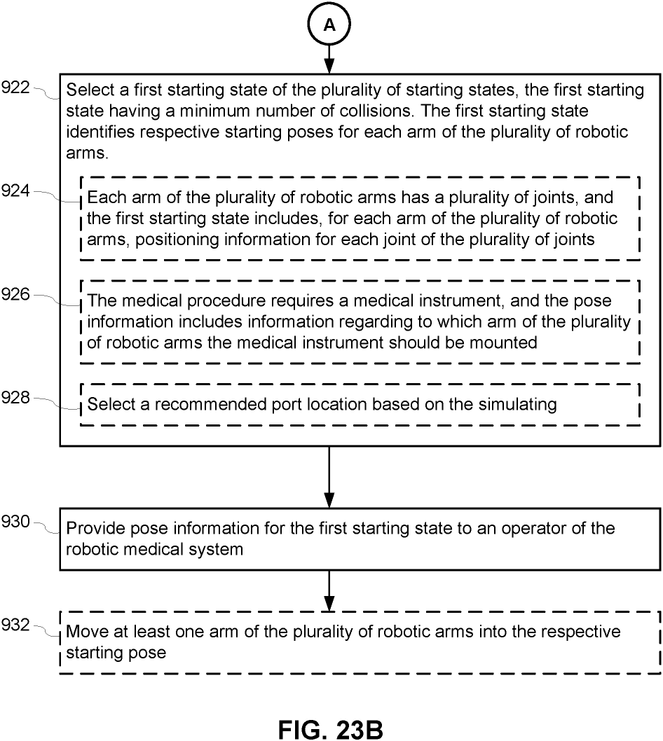

FIGS. 23A and 23B are a flowchart illustrating a method 900 for determining a starting state of a robotic medical system in accordance with some embodiments. The method 900 is performed at a robotic medical system (e.g., the medical system 36 or robotic medical system 300) having one or more processors (e.g., the processor(s) 280), memory (e.g., the memory 282), and a plurality of robotic arms. In some embodiments, the memory (e.g., the memory 282) stores instructions for execution by the one or more processor (e.g., the processor(s) 280). In some embodiments, at least a subset of the robotic arms are coupled to respective medical instruments.

The robotic medical system obtains (902) imaging data of a patient. For example, the robotic medical system obtains the imaging data as described above with respect to operation 604. In some embodiments, the imaging data includes a 3D surface scan of the patient. In some embodiments, the robotic medical system includes an imager (e.g., the 3-D scanner 704). In some embodiments, the imager is not part of the robotic medical system but is in communication with the robotic medical system. In some embodiments, the imager incorporates at least one of: time-of-flight or dot pattern recognition. In some embodiments, the robotic medical system uses the imaging data to identify one or more bony landmarks of the patient (e.g., a ribcage or hip bone).

In some embodiments, the imaging data for the patient includes (904) images of the patient after the patient is prepared for the medical procedure. In some embodiments, the imaging data for the patient includes images of the patient with a body cavity insufflated. In some embodiments, the system determines (e.g., in real time) one or more physical characteristics (e.g., size and/or other characteristics such as unique anatomy) of the patient. In some embodiments, the imaging data for the patient includes images of the patient after they have been arranged in position for the medical procedure (e.g., arranged on the patient support platform 302). In some embodiments, the imaging data includes a portion of a robotic arm and is used to localize the patient relative to the robotic arm (e.g., as described above with respect to operation 606).

The robotic medical system obtains (906) information on a medical procedure for the patient. For example, the robotic medical system obtains information on the medical procedure as described above with respect to operation 610. As another example, a physician or assistant informs the system of the type of medical procedure being performed (e.g., inputs the medical procedure details in the console 31). In some embodiments, the information on the medical procedure includes a type of procedure to be performed. In some embodiments, the information on the medical procedure includes information regarding additional equipment or tools to be used during the medical procedure (e.g., stirrups, liver retractors, arm boards, and/or anesthesia accessories).

The robotic medical system simulates (908), for each starting state of a plurality of starting states, the medical procedure from the starting state to identify a number of collisions occurring with arms of the plurality of robotic arms. The simulating uses the imaging data and the information on the medical procedure. For example, the simulating includes simulating robotic arm movements as described with respect to FIGS. 22B-22H. In some embodiments, the medical procedure involves a number of sequential steps and at least a subset of the steps have corresponding robotic arm position requirements. In some embodiments, simulating the medical procedure including simulating robotic arm movements between required robotic arm positions. In some embodiments, the simulating includes simulating alternative movements between required robotic arm positions to identify which alternative results in the least collisions. In some embodiments, the simulating is further based on a type of the robotic medical system (e.g., how many arms the system has and how they are mounted). In some embodiments, the simulating is further based on a type of robotic arm (e.g., how many joints the robotic has and what degrees of freedom are available). For example, in some embodiments, the robotic arm support has four degrees of freedom, the robotic arm has five degrees of freedom (e.g., roll and twist), and a medical instrument attached to the robotic arm has four degrees of freedom (e.g., roll, pitch, yaw, and insertion). In some embodiments, the simulating includes determining a number of the robotic arms of the system to be used in the medical procedure (e.g., determine whether a subset of the robotic arms won’t be used and can be stowed during the medical procedure).

In some embodiments, the simulating includes: (i) identifying a first set of port placements, (ii) determining starting poses based on the first set of port placements, and (iii) mapping trajectories of the robotic arms during the medical procedure, as the robotic arms move from the starting poses to positions required for different steps of the procedure. In some embodiments, the simulating includes: (i) repeating the identifying, determining, and mapping for a plurality of sets of port placements and a plurality of starting poses; and (ii) selecting the set of port placements and starting poses that result in a minimum number of collisions.

In some embodiments, the robotic medical system obtains (910) an anatomic target for the medical procedure, and the simulating further uses the anatomic target. For example, the robotic medical system obtains the anatomic target as described above with respect to operation 610. In some embodiments, a user inputs the medical procedure and target anatomy.

In some embodiments, the robotic medical system identifies (912) the anatomical target using the imaging data of the patient. For example, the robotic medical system identifies the anatomic target as described above with respect to operation 608. In some embodiments, the robotic medical system estimates locations of one or more key anatomical structures, such as relevant organs, based on the imaging data. In some embodiments, the anatomical target includes an external anatomical marker such as the navel, or an internal anatomical marker (e.g., the xiphoid process or the anterior superior iliac spine) that can be inferred based on the imaging data. In some embodiments, the system compares the imaging data of the patient with generalized imaging data (e.g., heuristics data, prior imaging data, imaging data from a database, etc.) and identifies the analytical target in accordance with the comparison. In some embodiments, the system estimates a position of an internal organ based on the anatomical target.

In some embodiments, the robotic medical system obtains (914) imaging data for environs of the robotic medical system, and the simulating further uses the imaging data for the environs. For example, the robotic medical system determines one or more boundary conditions (e.g., spatial limits) for the robotic arm. In some embodiments, the data includes visual data (e.g., the imaging data) of at least a portion of the patient and an environment (e.g., spaces and/or objects) surrounding the patient. In some embodiments, the environs include non-robotic components, such as bed-side accessories that clamp on to the system (e.g., liver retractors and anesthesia equipment that may be clamped to a bed and draped over the patient’s head). In some embodiments, the environs include moveable and removable (optional) portions of the patient support platform, e.g., moveable or removable leg supports. In some embodiments, based on the visual data of the environs, the robotic medical system identifies one or more boundary conditions or “keep out” zones for the robotic arms (e.g., to prevent collisions between the robotic arms and objects in the environs). In some embodiments, the robotic medical system uses one or more machine learning techniques to identity the non-robotic components and/or moveable and removable portions of the patient support platform (e.g., object recognition algorithm(s)).

In some embodiments, the robotic medical system obtains (918) positional information for one or more objects (or entities) in a vicinity of the robotic medical system, and the simulating further uses the positional information. For example, the positional information captures size (e.g., dimensions), shape, and/or depth information of at least a portion of the patient and one or more objects surrounding the patient (e.g., light fixtures, oxygen tanks, carts, displays, and trays). The positional information includes dimensional information (e.g., information such as distance, angle, etc.) that can be used to determine relative separations (e.g., spatial separation, angular separation) between the patient and other objects. In some embodiments, the objects include accessories (e.g., mounted on the bedside or on robotic arms, etc.) such as stirrups, liver retractors, arm boards, anesthesia accessories, and the like.

In some embodiments, the robotic medical system maps (920) trajectories for each arm of the plurality of robotic arms during the medical procedure and identifies collisions based on the trajectories. For example, the robotic medical system maps the trajectories to identify collisions between the patient and a robotic arm, between two robotic arms, between two instruments, between a robotic arm and an instrument, and/or between a robotic arm and another object in the vicinity.

The robotic medical system selects (922) a first starting state of the plurality of starting states, the first starting state having a minimum number of collisions. The first starting state identifies respective starting poses for each arm of the plurality of robotic arms. For example, the robotic medical system generates the optimized pose information and port placements as described above with respect to operation 614. In some embodiments, selecting the first starting state includes identifying how many of the robotic arms are to be used for the medical procedure (e.g., by simulating the use of different numbers of robotic arms for performing the medical procedure). In some embodiments, the robotic medical system selects the first starting state in accordance with it requiring a minimum number of the robotic arms.

In some embodiments, each arm of the plurality of robotic arms has (924) a plurality of joints, and the first starting state includes, for each arm of the plurality of robotic arms, positioning information for each joint of the plurality of joints. In some embodiments, the first starting state includes positioning information for each robotic arm and each robotic arm support (e.g., the arm support 105 or 320). In some embodiments, the plurality of joints includes a subset of joints that lock in place during the medical procedure and the positioning information includes locking positions for the subset of joints. For example, a robotic arm may include: (i) a set-up arm (e.g., the proximal joints of the arm, such as joints 404-1 through 404-6) used to position a surgical end of the arm, and (ii) a distal end of the surgical end of the arm. In some embodiments, the set-up joints remain in the locking positions (identified in the positioning information) during the medical procedure.

In some embodiments, the medical procedure requires (926) a medical instrument, and the pose information includes information regarding to which arm of the plurality of robotic arms the medical instrument should be mounted. In some embodiments, the medical instrument has different size options, and the pose information includes information on a recommended size option for the medical instrument. In some embodiments, the robotic medical system identifies an assigned task for each robotic arm (e.g., holding a cannula, a light, a camera, etc.). For example, a medical procedure requires three different medical instruments and the system identifies which arm holds each instrument (e.g., so as to minimize a number of collisions during the procedure).

In some embodiments, the robotic medical system selects (928) a recommended port location based on the simulating (e.g., as illustrated in FIGS. 22A and 22E). The recommended port location is a location (e.g., position) on the patient’s body through which a medical tool/instrument is inserted and constrained in motion. In some embodiments, the port location corresponds to an incision point (or an incision region) that is made through the skin of the patient to facilitate the medical procedure. In some embodiments, the port location corresponds to a natural orifice, such as a mouth of the patient (e.g., for a bronchoscopy procedure).

The robotic medical system provides (930) pose information for the first starting state to an operator of the robotic medical system. For example, the robotic medical system provides the pose information as described above with respect to operation 616. In some embodiments, the system notifies a user of an optimal pre-docking pose for one or more arms and provides the user (e.g., a hospital assistant) with an option to move the one or more arms to the respective optimal poses. In some embodiments, the robotic medical system displays one or more graphical representations indicating the pose information on a display (e.g., the touchscreen 26) or provides electrical signals to a display for displaying the pose information (e.g., displayed in VR or AR).

In some embodiments, the robotic medical system moves (932) at least one arm of the plurality of robotic arms into the respective starting pose. In some embodiments, the arms are moved into the starting poses from a draping pose. In some embodiments, the robotic medical system automatically drives to the first starting state. In some embodiments, the robotic medical system automatically docks to a cannula via integrated sensing mechanisms. In some embodiments, a user completes the docking manually after movement of the plurality of robotic arms into the respective starting poses.

In some embodiments, the arms are moved into the starting poses from a stowed position (e.g., under the table for the medical procedure). In some embodiments, the robotic arms are initially in a stowed position below a table top of the robotic medical system (e.g., as illustrated in FIG. 6). In some embodiments, the robotic arms are initially in a stowed position adjacent to a patient support platform (e.g., as illustrated in FIG. 16). In some embodiments, the arms are deployed into an initial drape pose whereby they can be draped to maintain a sterile boundary. In some embodiments, the system moves the robotic arms from the drape pose to the respective starting poses (e.g., based on the 3-D scanning data and surgical procedure information).

In some embodiments, the robotic medical system includes: (i) a plurality of robotic arms; (ii) one or more processors; and (iii) memory storing instructions that, when executed by the one or more processors, cause the one or more processors to: (a) obtain imaging data of a patient; (b) obtain information on a medical procedure for the patient; (c) for each starting state of a plurality of starting states, simulate the medical procedure from the starting state to identify a number of collisions occurring with arms of the plurality of robotic arms, the simulating using the imaging data and the information on the medical procedure; (d) select a first starting state of the plurality of starting states, the first starting state having a minimum number of collisions, where the first starting state identifies respective starting poses for each arm of the plurality of robotic arms; and (e) provide pose information for the first starting state to an operator of the robotic medical system.

In some embodiments, the robotic medical system includes an adjustable bar to which at least a subset of the arms is coupled. In some embodiments, each robotic arm is coupled to an instrument for performing a medical procedure on a patient. In some embodiments, the robotic medical system includes multiple robotic arms (e.g., four, five, or six arms) to provide increased flexibility for the surgeon (e.g., requiring careful setup and positioning to avoid intraoperative collisions while still being able to reach the target anatomy).

In some embodiments, the robotic medical system displays one or more graphical representations indicating the pose information on a display or provides electrical signals to a display for displaying the pose information (e.g., displayed in VR or AR).

In some embodiments, the memory further includes instructions that, when executed by the one or more processors, cause the one or more processors to obtain an anatomic target for the medical procedure. The simulating further uses the anatomic target. In some embodiments, the memory further includes instructions that, when executed by the one or more processors, cause the one or more processors to identify the anatomical target using the imaging data of the patient. In some embodiments, the memory further includes instructions that, when executed by the one or more processors, cause the one or more processors to obtain imaging data for environs of the robotic medical system, where the simulating further uses the imaging data for the environs. In some embodiments, the memory further includes instructions that, when executed by the one or more processors, cause the one or more processors to obtain positional information for one or more objects in a vicinity of the robotic medical system, where the simulating further uses the positional information.

In some embodiments, the memory further includes instructions that, when executed by the one or more processors, cause the one or more processors to select a recommended port location based on the simulating. In some embodiments, each arm of the plurality of robotic arms has a plurality of joints, and the first starting state includes, for each arm of the plurality of robotic arms, positioning information for each joint of the plurality of joints. In some embodiments, the memory further includes instructions that, when executed by the one or more processors, cause the one or more processors to move at least one arm of the plurality of robotic arms into the respective starting pose.

### 3 Implementing Systems and Terminology

FIG. 24 is a schematic diagram illustrating electronic components of a system 279 (e.g., a robotic medical system) in accordance with some embodiments. In some embodiments, the system 279 is an instance of one of the systems 10, 36, 47, 100, and 140A. The system 279 includes one or more processors 380, which are in communication with a computer-readable storage medium of memory 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 19, 23A, and 23B).

The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controller 386 are integrated with the input/output controller 384 and the input/output controller 384 provides control signals directly to the one or more actuators 387 (without a separate actuator controller). Although FIG. 24 shows that there is one actuator controller 386 (e.g., one actuator controller for the entire mobile medical platform, in other embodiments, additional actuator controllers are used (e.g., one actuator controller for each actuator, etc.). In some embodiments, the one or more processors 380 are in communication with one or more displays 381 for displaying information (e.g., recommended robotic arm poses and port locations) as described herein.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code, or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database, or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

What is claimed is:

1. A robotic medical system comprising:
a plurality of robotic arms;
one or more processors; and
memory storing instructions that, when executed by the one or more processors, cause the one or more processors to:
obtain imaging data of a patient;
obtain information on a medical procedure for the patient;
for each starting state of a plurality of starting states, simulate the medical procedure from the starting state to identify a number of collisions occurring with arms of the plurality of robotic arms, the simulating using the imaging data and the information on the medical procedure;
select a first starting state of the plurality of starting states, the first starting state having a minimum number of collisions, wherein the first starting state identifies respective starting poses for each arm of the plurality of robotic arms; and
provide pose information for the first starting state to an operator of the robotic medical system.

2. The robotic medical system of claim 1, wherein the memory further includes instructions that, when executed by the one or more processors, cause the one or more processors to obtain an anatomic target for the medical procedure, wherein the simulating further uses the anatomic target.

3. The robotic medical system of claim 2, wherein the memory further includes instructions that, when executed by the one or more processors, cause the one or more processors to identify the anatomical target using the imaging data of the patient.

4. The robotic medical system of claim 1, wherein the simulating comprises:
mapping trajectories for each arm of the plurality of robotic arms during the medical procedure; and
identifying collisions based on the trajectories.

5. The robotic medical system of claim 1, wherein the memory further includes instructions that, when executed by the one or more processors, cause the one or more processors to select a recommended port location based on the simulating.

6. The robotic medical system of claim 1, wherein each arm of the plurality of robotic arms has a plurality of joints, and wherein the first starting state includes, for each arm of the plurality of robotic arms, positioning information for each joint of the plurality of joints.

7. The robotic medical system of claim 1, wherein the memory further includes instructions that, when executed by the one or more processors, cause the one or more processors to obtain imaging data for environs of the robotic medical system, wherein the simulating further uses the imaging data for the environs.

8. The robotic medical system of claim 1, wherein the memory further includes instructions that, when executed by the one or more processors, cause the one or more processors to obtain positional information for one or more objects in a vicinity of the robotic medical system, wherein the simulating further uses the positional information.

9. The robotic medical system of claim 1, wherein the medical procedure requires a medical instrument, and wherein the pose information includes information regarding to which arm of the plurality of robotic arms the medical instrument should be mounted.

10. The robotic medical system of claim 1, wherein the memory further includes instructions that, when executed by the one or more processors, cause the one or more processors to move at least one arm of the plurality of robotic arms into the respective starting pose.

11. The robotic medical system of claim 1, wherein the imaging data for the patient includes images of the patient after the patient is prepared for the medical procedure.

12. A method for determining a starting state of a robotic system that includes a plurality of robotic arms, the method comprising:
obtaining imaging data of a patient;
obtaining information on a medical procedure for patient;
for each starting state of a plurality of starting states, simulating the medical procedure from the starting state to identify a number of collisions occurring with arms of the plurality of robotic arms, the simulating using the imaging data and the information on the medical procedure;
selecting a first starting state of the plurality of starting states, the first starting state having a minimum number of collisions, wherein the first starting state identifies respective starting poses for each arm of the plurality of robotic arms; and
providing pose information for the first starting state to an operator of the robotic system.

13. The method of claim 12, further comprising obtaining an anatomic target for the medical procedure, wherein the simulating further uses the anatomic target.

14. The method of claim 12, wherein the simulating comprises:
mapping trajectories for each arm of the plurality of robotic arms during the medical procedure; and
identifying collisions based on the trajectories.

15. The method of claim 12, further comprising selecting a recommended port location based on the simulating.

16. The method of claim 12, further comprising obtaining imaging data for environs of the robotic system, wherein the simulating further uses the imaging data for the environs.

17. The method of claim 12, further comprising obtaining positional information for one or more objects in a vicinity of the robotic system, wherein the simulating further uses the positional information.

18. The method of claim 12, wherein the medical procedure requires a medical instrument, and wherein the pose information includes information regarding to which arm of the plurality of robotic arms the medical instrument should be mounted.

19. The method of claim 12, further comprising moving at least one arm of the plurality of robotic arms into the respective starting pose.

20. The method of claim 12, wherein the imaging data for the patient includes images of the patient after the patient is prepared for the medical procedure.
